(19) United States
(12) Patent Application Publication
(10) Pub. No.: US 2025/0195548 A1
(43) Pub. Date: Jun. 19, 2025

(54) NEW USE

(71) Applicant: KANCERA AB, Solna (SE)

(72) Inventors: Thomas Olin, Solna (SE); Ioakim Spyridopoulos, Newcastle upon Tyne (GB)

(21) Appl. No.: 18/924,797

(22) Filed: Oct. 23, 2024

(30) Foreign Application Priority Data
Dec. 19, 2023 (GB) 2319559.7
Jan. 31, 2024 (GB) 2401289.0

(51) Int. Cl. A61K 31/675; A61K 31/4365; A61K 31/4465; A61K 31/519; A61K 31/616; A61K 31/727; A61P 9/10

(52) U.S. Cl. CPC A61K 31/675; A61K 31/4365; A61K 31/4465; A61K 31/519; A61K 31/616; A61K 31/727; A61P 9/10

(57) ABSTRACT

There is provided herein a compound of formula I, or pharmaceutically acceptable salt thereof,

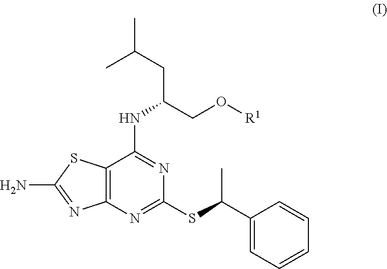

wherein R¹ is as defined herein, for use in the prevention of thrombus formation and/or thrombus growth and/or in the prevention of haemorrhage. In particular, the compound is for use in the prevention of left ventricular thrombus formation and/or thrombus growth and/or intramyocardial haemorrhage in myocardial infarction patients and particularly in conjunction with standard antiplatelet and anticoagulant medications.

### FIELD OF THE INVENTION

The present invention relates to a novel use of compounds that are known to be antagonists of the fractalkine receptor. In particular, the present invention relates to the use of such compounds, and compositions comprising the same, in methods for the prevention of thrombus formation and/or thrombus growth. In particular, the compounds are useful in the prevention of left ventricular thrombus and/or left ventricular thrombus growth in ST-elevation myocardial infarction patients. The compounds have also been found to prevent intramyocardial haemorrhage, which, in combination with the capacity of the compounds to prevent thrombus formation suggests that the compounds could provide improved protection of vital organ function in patients undergoing reperfusion treatment following myocardial infarction or large vessel occlusion stroke and for patients with heart failure.

### BACKGROUND OF THE INVENTION

The listing or discussion of an apparently prior-published document in this specification should not necessarily be taken as an acknowledgement that the document is part of the state of the art or is common general knowledge.

The compound, (2R)-2-[(2-amino-5-{[(1S)-1-phenylethyl]thio}[1,3]thiazolo[4,5-d]pyrimidin-7-yl)amino]-4-methylpentan-1-ol (A), and phosphate derivatives thereof, including (2R)-2-[(2-Amino-5-{[(1S)-1-phenylethyl]sulfanyl}[1,3]thiazolo[4,5-d]pyrimidin-7-yl)amino]-4-methylpentyl dihydrogen phosphate (B), are known to act as antagonists of the fractalkine receptor (CX3CR1) (Karlström et al. J. Med. Chem., 2013, 56, 3177-3190; WO 2020/008064)

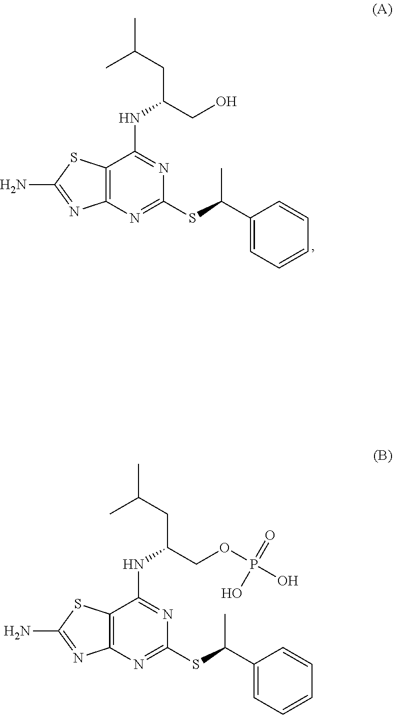 [SMILES: CC(C)C[C@H](CO)Nc1nc(S[C@@H](C)c2ccccc2)nc2nc(N)sc12 ; CC(C)C[C@H](COP(=O)(O)O)Nc1nc(S[C@@H](C)c2ccccc2)nc2nc(N)sc12]

Antagonism of the fractalkine receptor has been reported to have a range of potential clinical applications, including anti-inflammatory and anti-cancer effects.

Schaefer et al. Blood, 103 (2), 407-412 (2004) have reported that the fractalkine receptor CX3CR1 is present on human and rat platelets and suggested that it may play a role in platelet activation and adhesion. The report describes the possible role of the fractalkine axis in the formation of thrombus in the absence of anticoagulant and antiplatelet therapy.

Loh et al. J. Clin Med., 2023, 12, 4821, discusses the potential for fractalkine signalling being a relevant target in coronary artery disease, and mentions preclinical studies in which compound A (known as KAND567) has been found to reduce infarct size, inflammation and intramyocardial haemorrhage in a rat myocardial infarction model. However, this rat model did not involve any co-treatment with antiplatelet or anticoagulant therapies (Abelmoaty et al., Eur. Heart. J., 2019, 40, ehz746-0080), which would usually be the case in the relevant human populations. The recently completed FRACTAL (FRACTalkine inhibition in Acute myocardial infarction) study, which was designed to primarily investigate the safety and tolerability of the compound, in ST-elevation myocardial infarction patients undergoing percutaneous coronary intervention, is also described in this review article, but no results from the study are discussed.

Three broad factors are generally considered to contribute to blood clot (thrombus) formation, which are: hypercoagulability, hemodynamic changes and endothelial injury or dysfunction. Collectively, these factors are referred to as ‘Virchow's triad’.

Hypercoagulability refers to excessively easy clotting of blood, which may have a variety of causes, including inflammation, hyperviscosity, coagulation factor V Leiden mutation, coagulation factor II G2021A mutation, deficiency of antithrombin III, protein C or S deficiency, nephrotic syndrome, changes after severe trauma or burn, cancer, late pregnancy and delivery, race, advanced age, cigarette smoking, hormonal contraceptives, and obesity. Hemodynamic changes refers to alterations in normal blood flow, which may occur due to myocardial infarction, heart failure, venous stasis, long surgical operations, prolonged immobility and varicose veins. Hemodynamic changes may also be caused by heart failure and myocardial infarction. Endothelial injury and irritation may be caused by inflammation, ischemia-reperfusion, other trauma to blood vessels or contact with procoagulant surfaces, such as bacteria, medical devices or other foreign materials.

When blood clots become dislodged from the vascular wall, they can travel through the circulatory system and cause clotting and blockages (embolisms) in blood vessels, which can have fatal consequences. Blood clots forming in a vein and in the heart can travel to, and block an artery in, the lungs, causing a pulmonary embolism and blood clots forming in arteries can travel to any organ of the body causing a variety of medical conditions including myocardial infarction and stroke.

Blood clot formation in the left ventricle (‘left ventricular thrombus’) is a common complication of acute myocardial infarction, acute heart failure and dilated cardiomyopathy. In view of the risk, myocardial infarction patients are typically treated prophylactically with blood thinning medications, such as aspirin, P2Y₁₂ inhibitors and GPIIb/IIIa inhibitors, and anticoagulants, such as warfarin or heparin. However, despite these measures, the formation of a left ventricular thrombus remains a significant risk.

Accordingly, there remains a significant need for new antithrombotic medications. Particularly those that can have a significant effect in addition to treatment with conventional anticoagulant and antiplatelet medications, without causing a clinically relevant increase in the risk of bleeding or haemorrhage.

Another indication of future health risk for myocardial infarction patients is infarct size. When a myocardial infarction is suffered by a patient, an area of cardiac tissue is considered at risk of tissue death. The residual infarct size, as part of the area at risk, is usually not known until some time (such as around 90 days or more) after the initial infarction event. Patients with smaller residual infarct size tend to enjoy better long-term health and are at a lower risk of e.g. heart failure and cardiac death.

There is therefore a need for medication which will provide a likelihood of smaller residual myocardial infarct size following treatment.

### DETAILED DESCRIPTION OF THE INVENTION

It has now surprisingly been found that certain fractalkine receptor antagonists are able to significantly reduce the risk of thrombus formation. In particular, the fractalkine antagonists have been shown to reduce the rate of thrombus formation in acute myocardial infarction patients that are concomitantly being treated with established antiplatelet and anticoagulant medications, without causing a clinically relevant increase in the risk of bleeding or haemorrhage. On the contrary, the fractalkine antagonists have also been shown to decrease the incidence of intramyocardial haemorrhage and do not cause a clinically relevant increase in other bleeding.

It is highly surprising that these compounds can reduce the risk of thrombus formation on top of the effects achieved with established treatment with standard of care antiplatelet and anticoagulant medications, and also surprising that, as well as this effect, the compounds are able to reduce the risk of haemorrhaging in the presence of such medications. In clinical practice, when a physician is treating or preventing clot formation, agents with antithrombic and anticoagulant properties are used. Common and predictable side effects of such agents are often increased risk of bleeding. In view of the significant effects of established medications, a clinician would expect the compounds to have minimal additional effects on both blood clotting and haemorrhage.

Overall, this suggests that treatment with the compounds described herein can complement and/or override the effects of these established medications. These effects, alone and in combination, make the fractalkine receptor antagonists highly likely to have beneficial effects in a range of patients, including those with cardiovascular disorders including acute myocardial infarction (particularly ST-elevation myocardial infarction), heart failure and dilated cardiomyopathy. They are also likely to have beneficial protective effects in patients undergoing reperfusion therapy, such as ST-elevation myocardial infarction patients and large vessel occlusion stroke patients.

In a first aspect of the invention, there is provided a compound of formula I

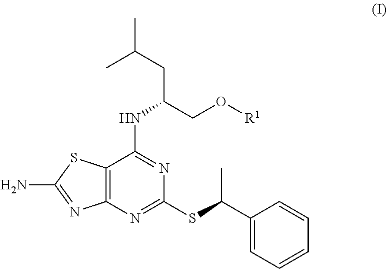

wherein R¹ is selected from hydrogen and —PO(OR²)(OR³),
wherein R² and R³ are each independently selected from the group consisting of hydrogen, C₁₋₄ alkyl and C₂₋₄ alkenyl, or a pharmaceutically acceptable salt thereof, f
for use in the prevention of thrombus formation and/or thrombus growth.

In an alternative first aspect of the invention, there is provided a method of preventing thrombus formation and/or thrombus growth comprising administering therapeutically effective amount of a compound of formula I, or pharmaceutically acceptable salt thereof, to a subject in need thereof.

In a further alternative first aspect of the invention, there is provided the use of a compound of formula I, or a pharmaceutically acceptable salt thereof for the manufacture of a medicament for the prevention of thrombus formation and/or thrombus growth.

Compound of formula I (including pharmaceutically acceptable salts thereof) may be referred to herein as the “compounds of the invention”.

For the avoidance of doubt, the skilled person will understand that references herein to compounds of particular aspects of the invention (such as the first aspect of the invention, i.e. referring to compounds of formula I as defined in the first aspect of the invention) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Pharmaceutically acceptable salts include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular acid addition salts that may be mentioned include those formed by reaction with corresponding acids, thus protonating the compound of the invention, to form carboxylate salts (e.g. formate, acetate, trifluoroacetate, propionate, isobutyrate, heptanoate, decanoate, caprate, caprylate, stearate, acrylate, caproate, propiolate, ascorbate, citrate, glucuronate, glutamate, glycolate, α-hydroxybutyrate, lactate, tartrate, phenylacetate, mandelate, phenylpropionate, phenylbutyrate, benzoate, chlorobenzoate, methylbenzoate, hydroxybenzoate, methoxybenzoate, dinitrobenzoate, o-acetoxy-benzoate, salicylate, nicotinate, isonicotinate, cinnamate, oxalate, malonate, succinate, suberate, sebacate, fumarate, malate, maleate, hydroxymaleate, hippurate, phthalate or terephthalate salts), hydrogen halide salts (e.g. chloride, bromide or iodide salts), sulphonate salts (e.g. benzenesulphonate, methyl-, bromo- or chloro-benzenesulphonate, xylenesulphonate, methanesulphonate, ethanesulphonate, propanesulphonate, hydroxy-ethanesulphonate, 1- or 2-naphthalene-sulphonate or 1,5-naphthalene-disulphonate salts) or sulphate, pyrosulphate, bisulphate, sulphite, bisulphite, phosphate, monohydrogenphosphate, dihydrogenphosphate, metaphosphate, pyrophosphate or nitrate salts, and the like.

Particular base addition salts that may be mentioned include salts formed by reaction with corresponding bases, thus removing a proton from compounds of the invention, to form salts with alkali metals (such as Na and K salts), alkaline earth metals (such as Mg and Ca salts), organic bases (such as ethanolamine, diethanolamine, triethanolamine and tromethamine) and inorganic bases (such as ammonia).

More particular salts that may be mentioned include Li, Na, K and ammonium salts (including monosalts and disalts). In particular, for compounds of the invention wherein R² and R³ each represent H particular salts that may be mentioned include diammonium salts, disodium salts, dilithium salts and dipotassium salts. For compounds of the invention in which R¹ represents H, a particular salt that may be mentioned is the hydrochloride salt.

For the avoidance of doubt, compounds of the invention may exist as solids, and thus the scope of the invention includes all amorphous, crystalline and part crystalline forms thereof, and may also exist as oils. Where compounds of the invention exist in crystalline and part crystalline forms, such forms may include solvates, which are included in the scope of the invention.

For the avoidance of doubt, compounds of the invention may also exist in solution (i.e. in solution in a suitable solvent). For example, compounds of the invention may exist in aqueous solution, in which case compounds of the invention may exist in the form of hydrates thereof.

Compounds of the invention may contain double bonds and, unless otherwise indicated, may thus exist as E (entgegen) and Z (zusammen) geometric isomers about each individual double bond. Unless otherwise specified, all such isomers and mixtures thereof are included within the scope of the invention.

Compounds of the invention may also exhibit tautomerism. All tautomeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

For the avoidance of doubt, the skilled person will understand that where a particular group is depicted herein as being bound to a ring system via a floating bond (i.e. a bond not shown as being bound to a particular atom within the ring), the relevant group may be bound to any suitable atom within the relevant ring system (i.e. the ring within which the floating bond terminates).

Unless otherwise specified, C_{1-z} alkyl groups (where z is the upper limit of the range) defined herein may be straight-chain or, when there is a sufficient number (i.e. a minimum of two or three, as appropriate) of carbon atoms, be branched-chain, and/or cyclic (so forming a C_{3-z} cycloalkyl group). When there is a sufficient number (i.e. a minimum of four) of carbon atoms, such groups may also be part cyclic (so forming a C_{4-z} partial cycloalkyl group). For example, cycloalkyl groups that may be mentioned include cyclopropyl and cyclobutyl. Similarly, part cyclic alkyl groups (which may also be referred to as “part cycloalkyl” groups) that may be mentioned include cyclopropylmethyl. When there is a sufficient number of carbon atoms, such groups may also be multicyclic (e.g. bicyclic or tricyclic) and/or spirocyclic. For the avoidance of doubt, particular alkyl groups that may be mentioned include straight chain (i.e. not branched and/or cyclic) alkyl groups.

Unless otherwise specified, C_{2-z} alkenyl groups (where z is the upper limit of the range) defined herein may be straight-chain or, when there is a sufficient number (i.e. a minimum of three) of carbon atoms, be branched-chain, and/or cyclic (so forming a C_{4-z} cycloalkenyl group). When there is a sufficient number (i.e. a minimum of five) of carbon atoms, such groups may also be part cyclic. For example, part cyclic alkenyl groups (which may also be referred to as “part cycloalkenyl” groups) that may be mentioned include cyclopentenylmethyl and cyclohexenylmethyl. When there is a sufficient number of carbon atoms, such groups may also be multicyclic (e.g. bicyclic or tricyclic) or spirocyclic. For the avoidance of doubt, particular alkenyl groups that may be mentioned include straight chain (i.e. not branched and/or cyclic) alkenyl groups.

For the avoidance of doubt, unless otherwise specified, groups referred to herein as “alkyl” and/or “alkenyl” will be taken as referring to the highest degree of unsaturation in a bond present in such groups. Alternatively, it may be particularly specified that that such groups will comprise only the degree of unsaturation specified (i.e. in one or more bond therein, as appropriate; e.g. in in one bond therein).

For the avoidance of doubt, references to polycyclic (e.g. bicyclic or tricyclic) groups (for example when employed in the context of heterocyclyl or cycloalkyl groups (e.g. heterocyclyl)) will refer to ring systems wherein at least two scissions would be required to convert such rings into a non-cyclic (i.e. straight or branched) chain, with the minimum number of such scissions corresponding to the number of rings defined (e.g. the term bicyclic may indicate that a minimum of two scissions would be required to convert the rings into a straight chain). For the avoidance of doubt, the term bicyclic (e.g. when employed in the context of alkyl groups) may refer to groups in which the second ring of a two-ring system is formed between two adjacent atoms of the first ring, to groups in which two non-adjacent atoms are linked by an alkyl (which, when linking two moieties, may be referred to as alkylene) group (optionally containing one or more heteroatoms), which later groups may be referred to as bridged, or to groups in which the second ring is attached to a single atom, which latter groups may be referred to as spiro compounds.

The present invention also embraces isotopically-labelled compounds of the present invention which are identical to those recited herein, but for the fact that one or more atoms are replaced by an atom having an atomic mass or mass number different from the atomic mass or mass number usually found in nature (or the most abundant one found in nature). All isotopes of any particular atom or element as specified herein are contemplated within the scope of the compounds of the invention. Hence, the compounds of the invention also include deuterated compounds, i.e. compounds of the invention in which one or more hydrogen atoms are replaced by the hydrogen isotope deuterium.

For the avoidance of doubt, in cases in which the identity of two or more substituents in a compound of the invention may be the same, the actual identities of the respective substituents are not in any way interdependent. In particular, R² and R³ may be the same or different and if both R² and R³ represent C₁₋₄ alkyl, the specific group that they represent may be the same or different.

For the avoidance of doubt, the skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

Particular compounds of the invention that may be mentioned include those in which R² and R³ each independently represent C₁₋₄ alkyl (such as C₁₋₃ alkyl, for example C₁₋₂ alkyl (e.g. methyl)) or H.

Further particular compounds of the invention that may be mentioned include those in which R² and R³ both represent H.

Further particular compounds of the invention that may be mentioned include those in which R² and R³ each represent C₁₋₄ alkyl (such as C₁₋₃ alkyl, for example C₁₋₂ alkyl (e.g. methyl)).

Further compound of the invention that may be mentioned include those in which one of R² and R³ represents C₁₋₄ alkyl (such as C₁₋₃ alkyl, for example C₁₋₂ alkyl (e.g. methyl)), and the other of R² and R³ represents H.

In particular embodiments of the invention, the compound of formula I is selected from the group consisting of:

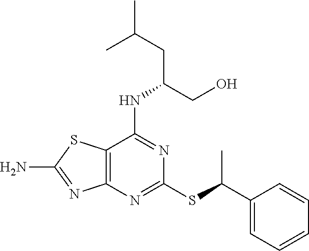 [SMILES: CC(C)C[C@H](CO)Nc1nc(S[C@@H](C)c2ccccc2)nc2nc(N)sc12]

(2R)-2-[(2-amino-5-{[(1S)-1-phenylethyl]thio}[1,3]thiazolo[4,5-d]pyrimidin-7-yl)amino]-4-methylpentan-1-ol;

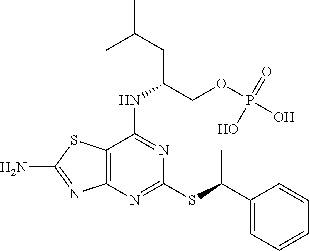 [SMILES: CC(C)C[C@H](COP(=O)(O)O)Nc1nc(S[C@@H](C)c2ccccc2)nc2nc(N)sc12]

(2R)-2-[(2-Amino-5-{[(1S)-1-phenylethyl]sulfanyl}[1,3]thiazolo[4,5-d]pyrimidin-7-yl)amino]-4-methylpentyl dihydrogen phosphate;

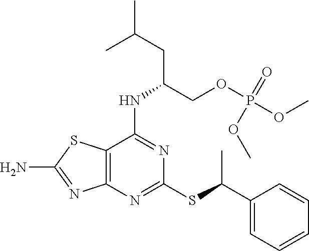 [SMILES: COP(=O)(OC)OC[C@@H](CC(C)C)Nc1nc(S[C@@H](C)c2ccccc2)nc2nc(N)sc12]

(2R)-2-[(2-Amino-5-{[(1S)-1-phenylethyl]sulfanyl}[1,3]thiazolo[4,5-d]pyrimidin-7-yl)amino]-4-methylpentyl dimethyl phosphate;

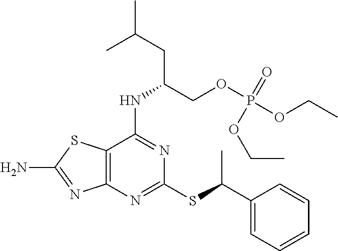 [SMILES: CCOP(=O)(OCC)OC[C@@H](CC(C)C)Nc1nc(S[C@@H](C)c2ccccc2)nc2nc(N)sc12]

(2R)-2-[(2-Amino-5-{[(1S)-1-phenylethyl]sulfanyl}[1,3]thiazolo[4,5-d]pyrimidin-7-yl)amino]-4-methylpentyl diethyl phosphate;

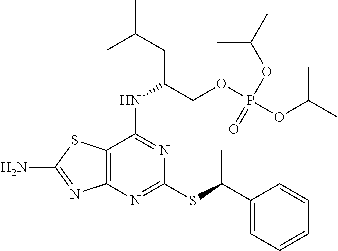

(2R)-2-[(2-Amino-5-{[(1S)-1-phenylethyl]sulfanyl}[1,3]thiazolo[4,5-d]pyrimidin-7-yl)amino]-4-methylpentyl bis(1-methylethyl) phosphate; and

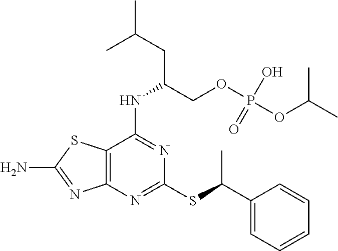

(2R)-2-[(2-Amino-5-{[(1S)-1-phenylethyl]sulfanyl}[1,3]thiazolo[4,5-d]pyrimidin-7-yl)amino]-4-methylpentyl 1-methylethyl hydrogen phosphate;
   or a pharmaceutically acceptable salt thereof.

In more particular embodiments of the invention, the compound of formula I is selected from the group consisting of:

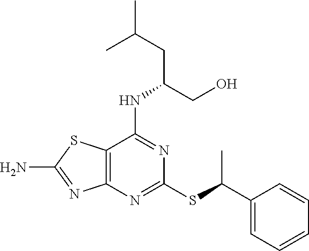 [SMILES: CC(C)C[C@H](CO)Nc1nc(S[C@@H](C)c2ccccc2)nc2nc(N)sc12]

(2R)-2-[(2-amino-5-{[(1S)-1-phenylethyl]thio}[1,3]thiazolo[4,5-d]pyrimidin-7-yl)amino]-4-methylpentan-1-ol; and

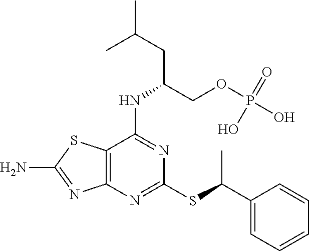 [SMILES: CC(C)C[C@H](COP(=O)(O)O)Nc1nc(S[C@@H](C)c2ccccc2)nc2nc(N)sc12]

(2R)-2-[(2-Amino-5-{[(1S)-1-phenylethyl]sulfanyl}[1,3]thiazolo[4,5-d]pyrimidin-7-yl)amino]-4-methylpentyl dihydrogen phosphate;
   or a pharmaceutically acceptable salt thereof.

In yet more particular embodiments, the compound of formula I is

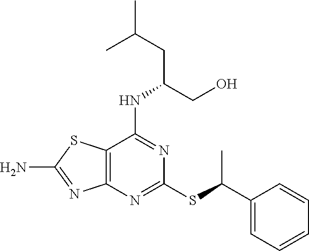 [SMILES: CC(C)C[C@H](CO)Nc1nc(S[C@@H](C)c2ccccc2)nc2nc(N)sc12]

(2R)-2-[(2-amino-5-{[(1S)-1-phenylethyl]thio}[1,3]thiazolo[4,5-d]pyrimidin-7-yl)amino]-4-methylpentan-1-ol, or a pharmaceutically acceptable salt thereof.

Particular compounds of the invention that may be mentioned include those compounds as described in the examples provided herein, and pharmaceutically acceptable salts thereof. For the avoidance of doubt, where such compounds of the invention include compounds in a particular salt form, compounds of the invention include those compounds in non-salt form and in the form of any pharmaceutically acceptable salt thereof (which may include the salt form present in such examples).

### Medical Uses

As described herein, compounds of formula I are useful in preventing thrombus formation and/or thrombus growth.

Although compounds of the invention may possess pharmacological activity as such, certain pharmaceutically-acceptable (e.g. “protected”) derivatives of compounds of the invention may exist or be prepared which may not possess such activity, but may be administered parenterally or orally and thereafter be metabolised in the body to form compounds of the invention. Such compounds (which may possess some pharmacological activity, provided that such activity is appreciably lower than that of the active compounds to which they are metabolised) may therefore be described as “prodrugs” of compounds of the invention.

As used herein, references to prodrugs will include compounds that form a compound of the invention, in an experimentally-detectable amount, within a predetermined time, following enteral or parenteral administration (e.g. oral or parenteral administration). All prodrugs of the compounds of the invention are included within the scope of the invention.

Furthermore, certain compounds of the invention may possess no or minimal pharmacological activity as such, but may be administered parenterally or orally, and thereafter be metabolised in the body to form compounds of the invention that possess pharmacological activity as such. Such compounds (which also includes compounds that may possess some pharmacological activity, but that activity is appreciably lower than that of the active compounds of the invention to which they are metabolised), may also be described as “prodrugs”.

In particular, as demonstrated in the patent applications WO 2020/008064 and WO 2021/224494, compounds of formula I in which R¹ represents-PO(OR²)(OR³) (and R² and R³ are as defined herein) may be metabolised in the body to form

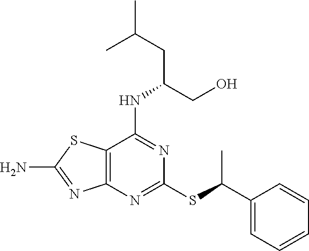 [SMILES: CC(C)C[C@H](CO)Nc1nc(S[C@@H](C)c2ccccc2)nc2nc(N)sc12]

(2R)-2-[(2-amino-5-{[(1S)-1-phenylethyl]thio}[1,3]thiazolo[4,5-d]pyrimidin-7-yl)amino]-4-methylpentan-1-ol (i.e. a compound of formula I in which R¹ represents H).

As used herein, the term prevention (and, similarly, preventing) will include references to the prophylaxis of the disease or disorder (and vice-versa) (in particular, the formation of thrombi (blood clots). As such, references to prevention may also be references to prophylaxis, and vice versa. In particular, such terms term may refer to achieving a reduction (for example, at least a 10% reduction, such as at least a 20%, 30% or 40% reduction, e.g. at least a 50% reduction) in the likelihood of the patient (or healthy subject) developing the condition (which may be understood as meaning that the condition of the patient changes such that patient is diagnosed by a physician as having, e.g. requiring treatment for, the relevant disease or disorder).

The compounds of the invention have been found to reduce the incidence of detectable thrombus formation. This may include preventing any formation of the thrombus and/or reducing the rate of thrombus formation so that it may dissolve through normal processes. By ‘preventing thrombus formation and/or thrombus growth’ we also include reducing thrombus formation and/or thrombus growth and reducing the rate of thrombus formation and/or thrombus growth.

In particular embodiments, the prevention of thrombus formation and/or thrombus growth is the prevention of thrombus formation.

In further particular embodiments, the prevention of thrombus formation and/or thrombus growth is prevention of thrombus growth.

As used herein, references to a patient (or to patients) will refer to a living subject being treated, including mammalian (e.g. human) patients. In particular, references to a patient will refer to human patients.

For the avoidance of doubt, the skilled person will understand that such prevention will be performed in a patient (or subject) in need thereof. The need of a patient (or subject) for such prevention may be assessed by those skilled the art using routine techniques.

As used herein, the terms disease and disorder (and, similarly, the terms condition, illness, medical problem, and the like) may be used interchangeably.

As used herein, the term effective amount will refer to an amount of a compound that confers a therapeutic effect on the treated patient. The effect may be observed in a manner that is objective (i.e. measurable by some test or marker) or subjective (i.e. the subject gives an indication of and/or feels an effect). In particular, the effect may be observed (e.g. measured) in a manner that is objective, using appropriate tests as known to those skilled in the art. For example, prevention of thrombus formation may be observed as a reduced incidence of thrombus formation in patients being treated with a compounds of formula I (or pharmaceutically acceptable salts thereof) compared to patients not being treated with compounds of formula I.

Thrombi may be detected using routine clinical tests and imaging methods. For example, thrombi may be detected using ultrasound, magnetic resonance imaging, venography, computed tomography and blood tests (e.g. the D-dimer blood test).

As used herein, the term thrombus (plural: thrombi) is used to refer to a blood clot formed in a blood vessel (vein or artery) and/or within the chambers of the heart.

Thrombi form as a result of coagulation of the blood and are typically comprised of aggregated platelets and red blood cells and a mesh of cross-linked fibrin protein.

Therapeutic agents that prevent (e.g. reduce the risk of) the formation of thrombi are known as antithrombotic agents.

Accordingly, in a further aspect of the invention, there is provided a compound of formula I (as defined herein), or a pharmaceutically acceptable salt thereof, for use as an antithrombotic agent.

There is also provided the use of compound of formula I (as defined herein), or a pharmaceutically acceptable salt thereof, in the manufacture of an antithrombotic medicament.

There is also provided a method of inducing an antithrombotic effect in a subject in need thereof comprising administering to the subject a therapeutically effective amount of a compound of formula I (as defined herein), or a pharmaceutically acceptable salt thereof.

As the compounds of the invention prevent thrombus formation and/or thrombus growth, they are also useful in the prevention of thrombosis, which refers to the formation of a thrombus (blood clot) in a blood vessel that obstructs the flow of blood through the circulatory system.

Accordingly, in a further aspect of the invention, there is provided a compound of formula I (as defined herein), or a pharmaceutically acceptable salt thereof, for use in the prevention of thrombosis.

There is also provided a method of preventing thrombosis comprising administering a therapeutically effective amount of a compound of formula I (as defined herein), or a pharmaceutically acceptable salt thereof, to a patient in need thereof.

There is also provided the use of a compound of formula I (as defined herein), or a pharmaceutically acceptable salt thereof, for the manufacture of a medicament for the prevention of thrombosis.

Similarly, the compounds of the invention can also prevent embolisms (a thrombosis forming in a vital organ, such as the lungs (pulmonary embolism)), preventing blood flow to the organ). In particular, the compounds of the invention may prevent systemic embolism and/or cardioembolic stroke.

Accordingly, in a further aspect of the invention, there is provided a compound of formula I (as defined herein), or a pharmaceutically acceptable salt thereof, for use in the prevention of embolism (such as systemic embolism and/or cardioembolic stroke).

There is also provided a method of preventing embolism (such as systemic embolism and/or cardioembolic stroke) comprising administering a therapeutically effective amount of a compound of formula I (as defined herein), or a pharmaceutically acceptable salt thereof, to a patient in need thereof.

There is also provided the use of a compound of formula I (as defined herein), or a pharmaceutically acceptable salt thereof, for the manufacture of a medicament for the prevention of embolism (such as systemic embolism and/or cardioembolic stroke).

In particular embodiments of the compounds for use, methods and uses described herein, the thrombus formation is in a patient with a cardiovascular disease.

In particular embodiments of the compounds for use, methods and uses described herein, the thrombus is in the heart (i.e. in a heart chamber or at the apex of the heart). That is the compounds prevent (e.g. reduce the risk of) the formation of a thrombus in the heart. More particularly, the thrombus is a left ventricular thrombus.

Thrombi generally form attached to the wall of a blood vessel or the inner walls of the heart. Such thrombi are referred to as ‘mural thrombi’.

Left ventricular thrombus is a common side effect of myocardial infarction (acute myocardial infarction), and typically involves the formation of a ‘mural thrombus’ attached to the wall of the left ventricle. The formation of a left ventricular thrombus can lead to the occurrence of a cardiac embolism in which the thrombus detaches from the ventricular wall, travels through the circulatory system and blocks a blood vessel.

Accordingly, in particular embodiments, the prevention of thrombus (e.g. left ventricular thrombus) formation and/or thrombus growth is in a patient with and/or being treated for a myocardial infarction (acute myocardial infarction). More particularly, the myocardial infarction is an ST-elevation myocardial infarction.

An ST-elevation myocardial infarction (also known as an ST segment elevation myocardial infarction (STEMI)) is a serious type of myocardial infarction in which there is a long interruption to the blood supply, which can cause significant damage to the heart. In particular, an ST-elevation myocardial infarction may involve the complete blockage of a major coronary artery supplying the heart. ST-elevation myocardial infarctions cause an increase of the ST segment of an echocardiogram (ECG). ST-elevation myocardial infarctions may also present themselves on the ECG with right bundle branch block or left bundle branch block with a QRS-duration of more than or equal to 0.12 seconds.

In further particular embodiments, the myocardial infarction is an anterior myocardial infarction. In particular embodiments, the ST-elevation myocardial infarction is an anterior STEMI with occlusion of the left anterior descending coronary artery.

More particularly, the myocardial infarction is an anterior myocardial infarction and the patient has a left ventricular ejection fraction of less than 50%.

Thrombus formation, and particularly left ventricular thrombus formation, are also common in patients with heart failure (particularly acute heart failure) and cardiomyopathy (e.g. dilated cardiomyopathy).

Thus, in further particular embodiments, the prevention of thrombus formation and/or thrombus growth is in a patient with heart failure (and, particularly, acute heart failure).

In further particular embodiments, the prevention of thrombus formation and/or thrombus growth is in a patient with cardiomyopathy (e.g. dilated cardiomyopathy).

In further particular embodiments, the prevention of thrombus formation and/or thrombus growth is in a patient undergoing reperfusion therapy. This applies particularly to the treatment of ST-elevation myocardial infarction and stroke (particularly large vessel occlusion stroke).

Yet more particularly, the prevention of thrombus (e.g. left ventricular thrombus) formation and/or thrombus growth is in a patient with and/or being treated for a myocardial infarction (acute myocardial infarction), wherein the patient has being treated with, or is being treated with percutaneous coronary intervention.

As used herein, ‘percutaneous coronary intervention’ is a minimally invasive procedure used to place a stent (or stents) in a blocked coronary artery, so restoring blood flow to the heart muscle. The procedure involves insertion of a catheter into a blood vessel in either the groin or arm, threading the catheter through the blood vessels into the narrowed coronary artery, then inflating a balloon tip covered with a stent inside the artery in order to expand the stent and compress the plaque in the narrowed artery, so restoring blood flow.

As mentioned herein, the compounds of the invention can also prevent thrombosis. Thus, in certain embodiments, the prevention of thrombus formation and/or thrombus growth further comprises the prevention of thrombosis. In such cases, the thrombus may alternatively be referred to as a thrombosis (for example an arterial thrombosis). This is particularly relevant to arterial thromboses, which are rich in platelets and dependent on platelet activation, and may therefore be susceptible to treatment with the compounds of the invention in view of their activity as antagonists of the fractalkine receptor.

In particular embodiments, the thrombus is an arterial thrombosis. In such embodiments, the prevention of thrombus formation may be termed the prevention of arterial thrombosis. In alternative embodiments, the prevention of thrombus formation and/or thrombus growth further comprises the prevention of thrombosis, wherein the thrombosis is an arterial thrombosis.

In further particular embodiments, the thrombus is a venous thrombosis (for example a deep vein thrombosis). In such embodiments, the prevention of thrombus formation may be termed the prevention of venous thrombosis. In alternative embodiments, the prevention of thrombus formation and/or thrombus growth further comprises the prevention of thrombosis, wherein the thrombosis is a venous thrombosis.

Thrombosis is often discussed with reference to ‘large vessel pathologies’ (for example stroke, myocardial infarction and venous/arterial thrombosis). However, ‘microvascular thrombosis’ (thromboses in the smallest blood vessels) is also associated with a range of disease states, and has been associated with the pathogenesis of organ injuries (for example in Covid-19 patients (Bray et al., Trans. Res., 2020, 225, 105-130).

The compounds' effects in preventing thrombus formation are believed to extend to the prevention of thrombi forming in the microvasculature, and therefore to, the prevention of microvascular thrombosis. Thus, in further particular embodiments, the thrombus is a microvascular thrombosis. In such embodiments, the prevention of thrombus formation may be termed the prevention of microvascular thrombosis. In alternative embodiments, the prevention of thrombus formation and/or thrombus growth further comprises prevention of thrombosis, wherein the thrombosis is a microvascular thrombosis.

In addition, it has been found that treatment with a compound of formula I, or a pharmaceutically acceptable salt thereof, can prevent/reduce the risk of haemorrhage (for example in myocardial infarction patients (including myocardial infarction patients having myocardial infarction with the specific features described in the various embodiments described herein)).

In particular embodiments, the hemorrhage is in the CNS or a vital organ.

In particular embodiments, the haemorrhage is intramyocardial haemorrhage.

As compounds of formula I, and pharmaceutically acceptable salts thereof, can prevent and/or reduce the risk of intramyocardial haemorrhage, they can also prevent and/or reduce the risk of disorders in which intramyocardial haemorrhage is a contributory factor, including mechanical rupture of the heart muscle, cardiogenic shock, acute heart failure and ventricular arrhythmias of the heart.

Accordingly, in certain embodiments, there is provided, a compound of formula I, or a pharmaceutically acceptable salt thereof, for use in preventing a disorder selected from mechanical rupture of the heart, cardiogenic shock, acute heart failure and ventricular arrhythmias of the heart. More particularly, the disorder is selected from mechanical rupture of the heart, cardiogenic shock and ventricular arrythmias of the heart.

In further embodiments, there is provided a compound of formula I, or a pharmaceutically acceptable salt thereof, for use in preventing mechanical rupture of the heart.

In further embodiments, there is provided a compound of formula I, or a pharmaceutically acceptable salt thereof, for use in preventing cardiogenic shock.

In further embodiments, there is provided a compound of formula I, or a pharmaceutically acceptable salt thereof, for use in preventing acute heart failure.

In further embodiments, there is provided a compound of formula I, or a pharmaceutically acceptable salt thereof, for use in preventing ventricular arrythmias of the heart.

In further particular embodiments that may be mentioned, there is provided a compound of formula I, or a pharmaceutically acceptable salt thereof, for use in preventing intramyocardial haemorrhage, wherein the use further comprises preventing a disorder selected from mechanical rupture of the heart, cardiogenic shock, acute heart failure and ventricular arrhythmias of the heart.

In further particular embodiments, the haemorrhage is a haemorrhagic stroke or haemorrhagic transformation of an ischemic stroke. In particular, the haemorrhage is a haemorrhagic stroke.

Without wishing to be bound by theory, the ability of the compounds of the invention to reduce haemorrhaging (and particularly intramyocardial haemorrhage) is consistent with an effect of reducing microvascular thrombosis, which is a common cause of haemorrhaging. It is believed that the compounds of the invention, could have this tissue protective effect in view of their cell interaction modulation capacity (for example modulation of the interactions between immune cells, endothelial cells and platelets) (an effect known to be associated with fractalkine receptor antagonism).

Thus, in particular embodiments of the compounds for use, methods and uses described herein, the compound of formula I, or pharmaceutically acceptable salt thereof, reduces the risk of haemorrhage (e.g. intramyocardial haemorrhage) (e.g. simultaneously with preventing/reducing the risk of thrombus formation (e.g. left ventricular thrombus formation).

In further particular embodiments, the compound of formula I, or pharmaceutically acceptable salt thereof is for use in preventing thrombus formation (e.g. left ventricular thrombus formation) and (simultaneously) preventing haemorrhage (e.g. intramyocardial haemorrhage), for example in a myocardial infarction patient (including myocardial infarction patients having myocardial infarction with the specific features described in the various embodiments described herein).

Thus, there is also provided a method of preventing thrombus formation (e.g. left ventricular thrombus formation) and (simultaneously) preventing haemorrhage (e.g. intramyocardial haemorrhage), comprising administering a therapeutically effective amount of a compound of formula I (as defined herein), or a pharmaceutically acceptable salt thereof, to a patient in need thereof (such as a myocardial infarction patient (including myocardial infarction patients having myocardial infarction with the specific features described in the various embodiments described herein).

There is further provided the use of a compound of formula I (as defined herein), or a pharmaceutically acceptable salt thereof, for the manufacture of a medicament for the preventing thrombus formation (e.g. left ventricular thrombus formation) and (simultaneously) preventing haemorrhage (e.g. intramyocardial haemorrhage), for example in a myocardial infarction patient (including myocardial infarction patients having myocardial infarction with the specific features described in the various embodiments described herein).

Compounds of the invention have also been found to prevent or suppress expansion of myocardial infarct size in myocardial infarction patients (including myocardial infarction patients having myocardial infarction with the specific features described in the various embodiments described herein (e.g. ST-elevation myocardial infarction). More particularly, the compounds of the invention prevent or suppress expansion of myocardial infarct size in patients without intramyocardial haemorrhage.

Accordingly, in further embodiments, the compounds are for use in preventing thrombus formation and/or thrombus growth and preventing haemorrhage (e.g. intramyocardial haemorrhage) and preventing or suppressing expansion of myocardial infarct size (for example in patients without intramyocardial haemorrhage).

As used herein, the term ‘infarct’ (e.g. myocardial infarct) may be understood to refer to an area of dead or dying tissue (e.g. heart tissue), resulting from an obstruction in the local blood supply.

As the compounds of the invention have been found to prevent haemorrhage, in a further aspect of the invention, there is provided a compound of formula I (as defined herein), or a pharmaceutically acceptable salt thereof, for use in preventing haemorrhage (e.g. intramyocardial haemorrhage) (e.g. in a myocardial infarction patient, (including myocardial infarction patients having myocardial infarction with the specific features described in the various embodiments described herein)). In particular embodiments of this aspect, the use further comprises preventing or suppressing expansion of myocardial infarct size.

In a further aspect of the invention, there is provided a method of preventing haemorrhage (e.g. intramyocardial haemorrhage), comprising administering a therapeutically effective amount of a compound of formula I (as defined herein), or a pharmaceutically acceptable salt thereof, to a patient in need thereof (such as a myocardial infarction patient (including myocardial infarction patients having myocardial infarction with the specific features described in the various embodiments described herein). In particular embodiments of this aspect, the method further comprises preventing or suppressing expansion of myocardial infarct size.

There is further provided the use of a compound of formula I (as defined herein), or a pharmaceutically acceptable salt thereof, for the manufacture of a medicament fo preventing haemorrhage (e.g. intramyocardial haemorrhage), for example in a myocardial infarction patient (including myocardial infarction patients having myocardial infarction with the specific features described in the various embodiments described herein). In particular embodiments of this aspect, the use further comprises preventing or suppressing expansion of myocardial infarct size.

In particular embodiments of these aspects of the invention, the prevention of haemorrhage (e.g. intramyocardial haemorrhage) further comprises preventing a disorder selected from mechanical rupture of the heart, cardiogenic shock, acute heart failure and ventricular arrhythmias of the heart. More particularly, the disorder is selected from mechanical rupture of the heart, cardiogenic shock and ventricular arrythmias of the heart.

In further particular embodiments of these aspects of the invention, the prevention of haemorrhage (e.g. intramyocardial haemorrhage) further comprises preventing or suppressing expansion of myocardial infarct size and preventing a disorder selected from mechanical rupture of the heart, cardiogenic shock, acute heart failure and ventricular arrhythmias of the heart. More particularly, the disorder is selected from mechanical rupture of the heart, cardiogenic shock and ventricular arrythmias of the heart.

As used herein, preventing or suppressing expansion of myocardial infarct size, may be understood to include reducing expansion (including reducing the rate of expansion) of myocardial infarct size and/or minimising expansion (including minimising the rate of expansion) of myocardial infarct size.

As mentioned above, the ability of the compounds to prevent thrombus formation and/or thrombus growth has the effect of preventing embolism (e.g. systemic embolism or cardioembolic stroke). These effects on thrombus formation/thrombus growth in combination with their effect of reducing haemorrhage make them particular suitable for these purposes.

Thus, in particular embodiments, the prevention of thrombus formation and/or thrombus growth (optionally in combination with the reduced risk of haemorrhage) further comprises the prevention of systemic embolism.

Thus, in particular embodiments, the prevention of thrombus formation and/or thrombus growth (optionally in combination with the reduced risk of haemorrhage) further comprises the prevention of cardioembolic stroke.

These combination of preventing left ventricular thrombus formation and/or growth and intramyocardial haemorrhage is believed to a have a beneficial protective effect on the myocardium after myocardial infarction.

Accordingly, in particular embodiments, the compounds of formula I, and pharmaceutically acceptable salts thereof, also reduce the risk of systemic embolism, adverse cardiac remodelling and heart failure.

Additionally, the combination of preventing thrombus formation and/or growth (e.g. left ventricular thrombus formation and intramyocardial haemorrhage suggests that the compounds of the invention may have a protective effect against reperfusion injury, for example in patients with ST-elevation myocardial infarction or large vessel occlusion stroke undergoing reperfusion therapy. In such patients, it is believed that the compounds of the invention may provide improved protection of vital organ function.

The skilled person will understand that reperfusion therapy refers to treatment to restore blood flow either through or around blocked arteries. Such reperfusion therapy may involve the use of thrombolytic (fibrinolytic) drugs or surgical methods, including percutaneous coronary intervention and (less commonly) coronary artery bypass surgery.

Thus, in particular embodiments of the invention, the patient is undergoing reperfusion therapy (for example for ST-elevation myocardial infarction or large vessel occlusion stroke). In more particular embodiments, the reperfusion therapy comprises the use of thrombolytic drugs. In further particular embodiments, the reperfusion therapy comprises surgical methods (for example percutaneous coronary intervention).

In yet further embodiments of the invention, the prevention of thrombus formation and/or thrombus growth (optionally in combination with the prevention of haemorrhage) further comprises the prevention of reperfusion injury.

In yet further embodiments of the invention, the prevention of thrombus formation and/or thrombus growth is in a patient that has been treated with or is being treated with a thrombectomy procedure. Such thrombectomy procedures may also provide another example of a reperfusion therapy.

In a further aspect of the invention, there is provided a compound of formula I, or a pharmaceutically acceptable salt thereof, as defined herein, for use in preventing or suppressing expansion of myocardial infarct size in a patient with and/or being treated for (e.g. with) myocardial infarction.

In an alternative further aspect of the invention, there is provided a method of preventing or suppressing expansion of myocardial infarct size in a patient in need thereof (e.g. a patient with and/or being treated for (e.g. with) myocardial infarction, comprising administering a therapeutically effective amount of a compound of formula I, or a pharmaceutically acceptable salt thereof, as defined herein.

In a further alternative further aspect of the invention, there is a provided the use of a compound of formula I, or a pharmaceutically acceptable salt thereof, as defined herein, for the manufacture of a medicament for the prevention or suppression of expansion of myocardial infarct size in a patient with and/or being treated for (e.g. with) myocardial infarction.

In particular embodiments of these aspects of the invention, the preventing or suppressing expansion of myocardial infarct size is in a patient without intramyocardial haemorrhage.

In further particular embodiments, the use or method further comprises preventing intramyocardial haemorrhage.

In further particular embodiments of these aspects of the invention, the myocardial infarction is an ST-elevation myocardial infarction.

By preventing (and prevention of) or suppressing (and suppression of) expansion of myocardial infarct size, we include achieving a clinically significant reduction in infarct size, for example as compared to myocardial infarct size in a patient not treated with a compound of formula I, or a pharmaceutically acceptable salt thereof, as defined herein (alone or in combination with other appropriate medications, such as the antiplatelet and anticoagulant medications described herein). For example, this may include achieving a 5% to 50% reduction in infarct size, such as a 5% to 30% reduction, or a 10% to 30% reduction (for example around a 20% reduction), as determined using appropriate techniques known to the person skilled in the art (such as those discussed herein, including in the Examples).

The eventual infarct size in a myocardial infarction patient is not usually known until some time after the initial myocardial infarction event. Accordingly, infarct size is most appropriately measured some time after the initial myocardial infarction, for example, at a time point of around 30 or more days (for example 30 to 150 days) after the initial myocardial infarction event, such as 60 or more days. More particularly infarct size may be measured after a period around 90 days or more following an initial myocardial infarction event. Additionally, or alternatively, infarct size may be measured by late gadolinium-enhanced (LGE) magnetic resonance imaging (MRI) or Echocardiography. Most particularly, infarct size may be measured by late-gadolinium enhanced magnetic resonance imaging.

The ability of the compounds of the invention to prevent intramyocardial haemorrhage and reduce infarct size are expected to reduce damage to the heart tissue resulting from myocardial infarction and therefore to preserve heart function, including left ventricular ejection fraction, which may be reduced as a result of myocardial infarction, particularly ST-elevation myocardial infarction (such as ST-elevation myocardial infarction with left anterior descendent artery involvement).

Thus, in particular embodiments of the methods and uses described herein the prevention of thrombus formation and/or thrombus growth in the heart of a patient (e.g. a myocardial infarction (particularly an ST-elevation myocardial infarction patient), including all particular aspects and embodiments of such uses and methods described herein, further comprises preserving left ventricular ejection fraction, preventing reduced left-ventricular ejection fraction or improving left ventricular ejection fraction.

Similarly, in particular embodiments of the methods and uses described herein the prevention of haemorrhage (and particularly intramyocardial haemorrhage) (e.g. a myocardial infarction (particularly an ST-elevation myocardial infarction patient), including all particular aspects and embodiments of such uses and methods described herein, further comprises preserving left ventricular ejection fraction, preventing reduced left-ventricular ejection fraction or improving left ventricular ejection fraction.

As described herein, preventing reduced left ventricular ejection fraction, may be understood to include that the left ventricular ejection fraction of the patient is maintained in the normal range for a patient of their age and fitness. In particular, following treatment, the left ventricular ejection fraction of the patient may be greater than about 50%.

As described herein, preserving left ventricular ejection fraction, may be understood to include maintaining the ejection fraction of the patient at the same, or a substantially similar level, as it was prior to myocardial infarction. It may also be understood to include maintaining ejection fraction at a higher level than would be achieved in the absence of treatment with a compound of formula I, or a pharmaceutically acceptable salt thereof (for example, if the patient were treated with one or more of the known anticoagulant and/or antiplatelet therapies described herein, or an appropriate combination thereof, such as the combination used in the clinical study described in the Examples included herein in the absence of treatment with a compound of formula I, or a pharmaceutically acceptable salt thereof).

As described herein, improving left ventricular ejection fraction, may be understood to include improving the ejection fraction of the patient relative to a patient not treated with a compound of formula I, or a pharmaceutically acceptable salt thereof (for example if the patient were treated with one or more of the known anticoagulant and/or antiplatelet therapies described herein, or an appropriate combination thereof, such as the combination used in the clinical study described in the Examples included herein in the absence of treatment with a compound of formula I, or a pharmaceutically acceptable salt thereof).

The skilled person will understand that the effects of treatments on heart function, and, in particular, left ventricular ejection fraction, are generally observed some time after the initial myocardial infarction, when the heart has had some time to recover. Most commonly, improvements will first be observed in tests done approximately 3 months after the myocardial infarction. Accordingly, in the context of the invention, preventing reduced left ventricular ejection fraction, preserving left ventricular ejection fraction and improving left ventricular ejection fraction may be understood to involve providing a measurable effect on heart function at the time point of 3 months after the myocardial infarction and/or at the time point of 1 year after the myocardial infarction.

Ejection fraction, and particularly left ventricular ejection fraction, may also be measured at a time point of around 30 or more days (for example 30 to 150 days) after the initial myocardial infarction event, such as 60 or more days. More particularly ejection fraction, including left ventricular ejection fraction, may be measured after a period around 90 days or more following an initial myocardial infarction event.

Ejection fraction, including left ventricular ejection fraction, may be determined using methods known to the skilled person, such as by echocardiogram or CT scan (in particular echocardiogram).

In a further aspect of the invention, there is provided a compound of formula I, or a pharmaceutically acceptable salt thereof, for use in preventing reduced left ventricular ejection fraction.

As described herein, the compounds of the invention are effective in the prevention of thrombus formation and/or thrombus growth and/or intramyocardial haemorrhage in patients having ST-elevation myocardial infarction, heart failure (particularly acute heart failure) and dilated cardiomyopathy.

In view of the previously-reported effects of the compounds in the treatment of cardiovascular disorders, in a further aspect of the invention, there is provided a compound of formula I, or a pharmaceutically acceptable salt thereof, as defined herein, for use in the treatment of a cardiovascular disease selected from the group consisting of ST-elevation myocardial infarction, heart failure (particularly acute heart failure), and dilated cardiomyopathy, wherein the treatment further comprises prevention of thrombus formation and/or thrombus growth.

In an alternative further aspect of the invention, there is provided a method of treating a cardiovascular disease selected from the group consisting of ST-elevation myocardial infarction, heart failure (particularly acute heart failure), and dilated cardiomyopathy, comprising administering a therapeutically effective amount of a compound of formula I, or a pharmaceutically acceptable salt thereof, as defined herein, to a patient need thereof, wherein the treatment further comprises prevention of thrombus formation and/or thrombus growth.

In another alternative further aspect of the invention, there is provided the use of a compound of formula I, or a pharmaceutically acceptable salt thereof, as defined in, for the manufacture of a medicament for the treatment of a cardiovascular disease selected from the group consisting of ST-elevation myocardial infarction, heart failure (particularly acute heart failure), and dilated cardiomyopathy, wherein the treatment further comprises prevention of thrombus formation and/or thrombus growth.

In particular embodiments of these aspects, the treatment further comprises prevention of intramyocardial haemorrhage.

In further particular embodiments of these aspects in relation to the treatment of ST-elevation myocardial infarction, the treatment, with the compounds of the invention, further comprises preventing or suppressing expansion of myocardial infarct size (for example in a patient without intramyocardial haemorrhage).

Combinations with Other Antithrombotic Agents

High risk patients, such as myocardial infarction patients, patients with (acute) heart failure or dilated cardiomyopathy and/or those undergoing reperfusion therapy (such as ST-elevation myocardial infarction patients and large vessel occlusion stroke patients, are routinely treated with a range of antithrombotic agents, often in combination. Accordingly, the compounds of formula I (including pharmaceutically acceptable salts thereof) may be used in combination with other antithrombotic agents, and such combinations may offer a substantial reduction in the occurrence of thrombus formation compared to existing therapies.

In fact, it has surprisingly been found that using the compounds of the invention in combination with ‘standard of care’ antiplatelet and anticoagulant medications can lead to surprising benefits in terms of both the ability of the compounds to prevent thrombus formation and/or thrombus growth and to prevent or reduce the likelihood of haemorrhage (such as intramyocardial haemorrhage).

With respect to preventing thrombus formation and/or thrombus growth, it is surprising that the compounds of the invention have been found to have an additional antithrombotic effect on top of existing medications, and, with respect to the ability of the compounds to prevent haemorrhage, it is highly surprising that the compounds can override the effects of existing antiplatelet and anticoagulant therapies.

Collectively, these effects mean that, when used with established anticoagulant and antiplatelet medications, the compounds of the invention are likely to offer significant benefits, including reducing the risk of systemic embolism and stroke, and of the other indications described herein.

Established antithrombotic agents fall into three categories: antiplatelet medications, anticoagulants and thrombolytic drugs.

Antiplatelet medications reduce the activation and aggregation of platelets and thereby inhibit thrombus formation. They are widely used as prophylactics of thrombotic diseases, including myocardial infarction and stroke. Various classes for antiplatelet medications exist, including adenosine diphosphate (ADP) receptor inhibitors (such as cangrelor, clopidogrel, prasugrel, ticagrelor and ticlopidine), adenosine reuptake inhibitors (such as dipyridamole), glycoprotein IIb/IIIa inhibitors (such as abciximab, eptifibatide and tirofiban), (irreversible) cyclooxygenase inhibitors (such as aspirin and tiflusal), phosphodiesterase inhibitors (such as cilostazol), protease-activated receptor-1 antagonists (such as vorapaxar), thromboxane inhibitors, thromboxane receptor antagonists and thromboxane synthase inhibitors. Dual antiplatelet therapy is also commonly used in patients at high risk of developing blood clots. Typically, this will be a combination of a cyclooxygenase inhibitor (e.g. aspirin) and a ADP receptor inhibitor (e.g. clopidogrel, prasugrel, ticagrelor, etc.).

Anticoagulants inhibit processes within the coagulation cascade, which occurs after platelet aggregation. Common classes of anticoagulants include vitamin K antagonists (such as warfarin), heparin (including unfractionated heparin and low molecular weight ‘fractionated’ heparins (LMHW)), synthetic pentasaccharide inhibitors of factor Xa (such as fondaparinux, idraparinux and idrabiotaparinux) and direct oral anticoagulants (DOACs), which include direct factor Xa inhibitors (such as rivarosaban, apixaban and edoxaban and betrixaban) and direct thrombin inhibitors (such as hirudin, lepirudin, bivalirudin, argatroban and dabigatran).

Thrombolytic agents breakdown blood clots after their formation (generally through fibrinolysis). Examples of approved thrombolytic (fibrinolytic) agents include streptokinase, urokinase and recombinant tissue plasminogen activators (such as alteplase, reteplase, tenecteplase and anistreplase).

In particular embodiments, the compound of formula I (as defined herein), or pharmaceutically acceptable salt thereof, is administered in conjunction with at least on antiplatelet medication.

In particular embodiments, the at least one antiplatelet medication is selected from the group consisting of a P2Y₁₂ receptor inhibitor (also known as adenosine diphosphate receptor inhibitors) (such as cangrelor, clopidogrel, prasugrel, ticagrelor or ticlopidine), an adenosine reuptake inhibitor such as dipyridamole), a glycoprotein IIb/IIIa inhibitor (such as abciximab, eptifibatide or tirofiban), an (irreversible) cyclooxygenase inhibitor (such as aspirin or tiflusal), a phosphodiesterase inhibitor (such as cilostazol), a protease-activated receptor-1 antagonist (such as vorapaxar), a thromboxane inhibitor, a thromboxane receptor antagonist and a thromboxane synthase inhibitor, and combinations thereof.

In further particular embodiments, the at least one antiplatelet medication is selected from the group consisting of a P2Y₁₂ receptor inhibitor (such as cangrelor, clopidogrel, prasugrel, ticagrelor or ticlopidine), an adenosine reuptake inhibitor such as dipyridamole), a glycoprotein IIb/IIIa inhibitor (such as abciximab, eptifibatide or tirofiban), an (irreversible) cyclooxygenase inhibitor (such as aspirin and tiflusal), a phosphodiesterase inhibitor (such as cilostazol), a protease-activated receptor-1 antagonist (such as vorapaxar), and combinations thereof.

In further particular embodiments, the at least one antiplatelet medication is selected from the group consisting of an P2Y₁₂ receptor inhibitor (such as cangrelor, clopidogrel, prasugrel, ticagrelor and ticlopidine), an (irreversible) cyclooxygenase inhibitor (such as aspirin and tiflusal), a glycoprotein IIb/IIIa inhibitor (such as abciximab, eptifibatide or tirofiban), and combinations thereof.

In further particular embodiments, the at least one antiplatelet medication comprises an (irreversible) cyclooxygenase inhibitor. More particularly, the (irreversible) cyclooxygenase inhibitor is aspirin.

In further particular embodiments, the at least one antiplatelet medication comprises an P2Y₁₂ receptor inhibitor (such as such as cangrelor, clopidogrel, prasugrel, ticagrelor and ticlopidine). More particularly, the P2Y₁₂ receptor inhibitor is prasugrel.

In further particular embodiments, the at least one antiplatelet medication comprises an (irreversible) cyclooxygenase inhibitor (e.g. aspirin) and/or (e.g. and) a P2Y₁₂ receptor inhibitor (such as such as cangrelor, clopidogrel, prasugrel, ticagrelor and ticlopidine (e.g. prasugrel)). More particularly, the at least one antiplatelet medication comprises an (irreversible) cyclooxygenase inhibitor (e.g. aspirin) and a P2Y₁₂ receptor inhibitor (such as such as cangrelor, clopidogrel, prasugrel, ticagrelor and ticlopidine (e.g. prasugrel)) (i.e. the patient is treated with dual antiplatelet therapy (such as, particularly, treatment with a combination of aspirin and prasugrel) in addition to treatment with a compound of formula I, or pharmaceutically acceptable salt thereof).

In further particular embodiments, in addition to treatment with an (irreversible) cyclooxygenase inhibitor (e.g. aspirin) and/or (e.g. and) a P2Y₁₂ receptor inhibitor (such as such as cangrelor, clopidogrel, prasugrel, ticagrelor and ticlopidine (e.g. prasugrel)), the patient is optionally further treated with a glycoprotein IIb/IIIa inhibitor (such as abciximab, eptifibatide or tirofiban (e.g. tirofiban)). Accordingly, in such embodiments, the one or more antiplatelet medication comprises with an (irreversible) cyclooxygenase inhibitor (e.g. aspirin) and/or (e.g. and) a P2Y₁₂ receptor inhibitor (such as such as cangrelor, clopidogrel, prasugrel, ticagrelor and ticlopidine (e.g. prasugrel)) and, optionally, a glycoprotein Iib/IIIa inhibitor (such as abciximab, eptifibatide or tirofiban (e.g. tirofiban)).

In further particular embodiments, the compound of formula I, or pharmaceutically acceptable salt thereof, is administered in conjunction with at least one anticoagulant.

In particular embodiments, the at least one anticoagulant is selected from the group consisting of a vitamin K antagonist (such as warfarin), a heparin (including unfractionated heparin and low molecular weight ‘fractionated’ heparins (LMHW) (e.g. dalteparin)), a synthetic pentasaccharide inhibitor of factor Xa (such as fondaparinux, idraparinux and idrabiotaparinux) a direct factor Xa inhibitor (such as rivarosaban, apixaban and edoxaban and betrixaban), a direct thrombin inhibitor (such as hirudin, lepirudin, bivalirudin, argatroban and dabigatran), and combinations thereof.

More particularly, the at least one anticoagulant is unfractionated heparin or a low molecular weight heparin (e.g. dalteparin (Fragmin®)).

Yet more particularly, the at least one anticoagulant is unfractionated heparin.

In particular embodiments that may be mentioned, the compound of formula I, or pharmaceutically acceptable salt thereof, is administered in conjunction with at least on antiplatelet medication (including all of the particular embodiments thereof described herein) and at least one anticoagulant (including all of the particular embodiments thereof described herein).

In more particular embodiments, the compound of formula I, or pharmaceutically acceptable salt thereof, is for use in the prevention of left ventricular thrombus (for example in a myocardial infarction patient) and is administered in conjunction with at least on antiplatelet medication (including all of the particular embodiments thereof described herein) and at least one anticoagulant (including all of the particular embodiments thereof described herein).

Yet more particularly, the compound of formula I, or pharmaceutically acceptable salt thereof, is for use in the prevention of left ventricular thrombus (for example in a myocardial infarction patient) and is administered in combination with (irreversible) cyclooxygenase inhibitor (e.g. aspirin) and/or (e.g. and) a P2Y₁₂ receptor inhibitor (such as such as cangrelor, clopidogrel, prasugrel, ticagrelor and ticlopidine (e.g. prasugrel)), an anticoagulant (such as a heparin, and, more particularly, unfractionated heparin) and, optionally, a glycoprotein IIb/IIIa inhibitor (such as abciximab, eptifibatide or tirofiban (e.g. tirofiban)).

In further particular embodiments, there is provided at least one antiplatelet medication (as described herein) for use in the prevention of thrombus formation (and the related indications described herein, including, particularly, left ventricular thrombus), wherein the at least one antiplatelet medication is administered in conjunction with a compound of formula I (as defined herein), or a pharmaceutically acceptable salt thereof.

In further particular embodiments, there is provided at least one antiplatelet medication (as described herein) for use in the prevention of thrombus formation (and the related indications described herein including, particularly, left ventricular thrombus), wherein the at least one antiplatelet medication is administered in conjunction with a compound of formula I (as defined herein), or a pharmaceutically acceptable salt thereof, and at least one anticoagulant medication (as defined herein).

In further particular embodiments, there is provided at least one anticoagulant (as described herein) for use in the prevention of thrombus formation (and the related indications described herein including, particularly, left ventricular thrombus), wherein the at least one anticoagulant is administered in conjunction with a compound of formula I (as defined herein), or a pharmaceutically acceptable salt thereof.

In further particular embodiments, there is provided at least one anticoagulant (as described herein) for use in the prevention of thrombus formation (and the related indications described herein including, particularly, left ventricular thrombus), wherein the at least one anticoagulant is administered in conjunction with a compound of formula I (as defined herein), or a pharmaceutically acceptable salt thereof, and at least one antiplatelet medication (as defined herein).

In yet further embodiments that may be mentioned, the compound of formula I, or pharmaceutically acceptable salt thereof is administered in conjunction with a thrombolytic agent (as described herein) (and optionally also in conjunction with at least one antiplatelet medication and at least one anticoagulant as described herein).

As used herein “administered in conjunction with” (and similarly “administration in conjunction with”) includes that respective active agents are administered sequentially, separately or simultaneously, as part of a medical intervention directed towards treatment of the relevant condition (e.g. the prevention of thrombus formation and related disorders described herein). Similarly, any references to concomitant treatment with different active ingredients (or similar), may be understood to indicate that the respective active ingredients are administered sequentially, separately or simultaneously, as part of a general medical intervention directed towards preventing (reducing the risk of) thrombus formation.

Thus, in relation to the present invention, the term “administration in conjunction with” (and similarly “administered in conjunction with”) includes that the two, or more, active ingredients (i.e. a compound of the invention and one or more of the other antithrombotic agents described herein (e.g. an antiplatelet medication and/or an anticoagulant) are administered (optionally repeatedly) either together, or sufficiently closely in time, to enable a beneficial effect for the patient, that is greater, over the course of the (preventative) treatment, than if either agent is administered (optionally repeatedly) alone, in the absence of the other component, over the same course of (preventative) treatment. Determination of whether a combination provides a greater beneficial effect may be achieved routinely by the skilled person.

Further, in the context of the present invention, the term “in conjunction with” includes that the relevant individual active ingredients may be administered (optionally repeatedly) prior to, after, and/or at the same time as, administration of the other component. When used in this context, the terms “administered simultaneously” and “administered at the same time as” includes instances where the individual doses of the compound of the invention and the additional compound for the treatment of [add disease/disorder], or pharmaceutically acceptable salts thereof, are administered within 48 hours (e.g. within 24 hours, 12 hours, 6 hours, 3 hours, 2 hours, 1 hour, 45 minutes, 30 minutes, 20 minutes or 10 minutes) of each other.

Where references are made to approved therapeutic agents, such as the antiplatelet medications and anticoagulants discussed herein, it may be understood that the approved drug may be used in any form in which it is available and approved for clinical use in the territory in question. In particular, the approved agents may be in the form of the free acid or base, or in the form of a pharmaceutically acceptable salt. The suitability of the relevant form of the drug may be determined routinely by the skilled practitioner (for example by reference to standard references works, such as a Pharmacopoeia relevant to the territory in question (e.g. the European Pharmacopoeia, the United States Pharmacopoeia, and the like)

### Pharmaceutical Compositions

As described herein, compounds of the invention are useful as pharmaceuticals. Such compounds may be administered alone or may be administered by way of known pharmaceutical compositions/formulations, comprising the compound of the invention in combination with one or more pharmaceutically acceptable excipient.

As used herein, the term pharmaceutically-acceptable excipients includes references to vehicles, adjuvants, carriers, diluents, pH adjusting and buffering agents, tonicity adjusting agents, stabilizers, wetting agents and the like. In particular, such excipients may include adjuvants, diluents or carriers.

Thus, in a further aspect of the invention, there is provided a pharmaceutical composition, comprising a compound of formula I (as defined herein), or a pharmaceutically acceptable salt thereof, for use in the prevention of thrombus formation (as defined herein, and also in the prevention of the other related disorders described herein).

The skilled person will understand that compounds and compositions as described herein will normally be administered orally, intravenously, subcutaneously, buccally, rectally, dermally, nasally, tracheally, bronchially, sublingually, intranasally, topically, by any other parenteral route or via inhalation, in a pharmaceutically acceptable dosage form. In particular, compounds and compositions described herein may be administered orally or intravenously.

Pharmaceutical compositions as described herein will include compositions in the form of tablets, capsules or elixirs for oral administration, suppositories for rectal administration, sterile solutions or suspensions for parenteral or intramuscular administration, and the like.

Thus, in particular embodiments, the pharmaceutical formulation is provided in a pharmaceutically acceptable dosage form, including tablets or capsules, liquid forms to be taken orally or by injection, suppositories, creams, gels, foams, inhalants (e.g. to be applied intranasally), or forms suitable for topical administration. For the avoidance of doubt, in such embodiments, compounds of the invention may be present as a solid (e.g. a solid dispersion), liquid (e.g. in solution) or in other forms, such as in the form of micelles.

For example, in the preparation of pharmaceutical formulations for oral administration, the compound may be mixed with solid, powdered ingredients such as lactose, saccharose, sorbitol, mannitol, starch, amylopectin, cellulose derivatives, gelatin, or another suitable ingredient, as well as with disintegrating agents and lubricating agents such as magnesium stearate, calcium stearate, sodium stearyl fumarate and polyethylene glycol waxes. The mixture may then be processed into granules or compressed into tablets.

Soft gelatin capsules may be prepared with capsules containing one or more active compounds (e.g. compounds of the first and, therefore, second and third aspects of the invention, and optionally additional therapeutic agents), together with, for example, vegetable oil, fat, or other suitable vehicle for soft gelatin capsules. Similarly, hard gelatine capsules may contain such compound(s) in combination with solid powdered ingredients such as lactose, saccharose, sorbitol, mannitol, potato starch, corn starch, amylopectin, cellulose derivatives or gelatin.

Dosage units for rectal administration may be prepared (i) in the form of suppositories which contain the compound(s) mixed with a neutral fat base; (ii) in the form of a gelatin rectal capsule which contains the active substance in a mixture with a vegetable oil, paraffin oil, or other suitable vehicle for gelatin rectal capsules; (iii) in the form of a ready-made micro enema; or (iv) in the form of a dry micro enema formulation to be reconstituted in a suitable solvent just prior to administration.

Liquid preparations for oral administration may be prepared in the form of syrups or suspensions, e.g. solutions or suspensions, containing the compound(s) and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

Solutions for parenteral administration may be prepared as a solution of the compound(s) (including pharmaceutically acceptable salts thereof) in a pharmaceutically acceptable solvent. These solutions may also contain stabilizing ingredients and/or buffering ingredients and are dispensed into unit doses in the form of ampoules or vials. Solutions for parenteral administration may also be prepared as a dry preparation to be reconstituted with a suitable solvent extemporaneously before use.

Depending on e.g. potency and physical characteristics of the compound of the invention (i.e. active ingredient), pharmaceutical formulations that may be mentioned include those in which the active ingredient is present in an amount that is at least 1% (or at least 10%, at least 30% or at least 50%) by weight. That is, the ratio of active ingredient to the other components (i.e. the addition of adjuvant, diluent and carrier) of the pharmaceutical composition is at least 1:99 (or at least 10:90, at least 30:70 or at least 50:50) by weight.

The skilled person will understand that compounds of the invention may be administered (for example, as formulations as described hereinabove) at varying doses, with suitable doses being readily determined by one of skill in the art. For example, when administered orally, treatment with such compounds may comprise administration of a formulations typically containing between about 0.01 μg to about 2000 mg, for example between about 0.1 μg to about 500 mg, or between 1 μg to about 100 mg (e.g. about 20 μg to about 80 mg), of the active ingredient(s). When administered intravenously, the most preferred doses will range from about 0.001 to about 10 μg/kg/hour during constant rate infusion. Advantageously, treatment may comprise administration of such compounds and compositions in a single daily dose, or the total daily dosage may be administered in divided doses of two, three or four times daily (e.g. twice daily with reference to the doses described herein, such as a dose of 25 mg, 50 mg, 100 mg or 200 mg twice daily).

As described herein, patients being treated for ST-elevation acute myocardial infarction (including those undergoing percutaneous coronary intervention), may be treated with intracoronary, intravenous and peroral (particularly intravenous and/or peroral) dosages of a compound of formula I, or a pharmaceutically acceptable salt thereof.

In particular, the patient may be treated by intravenous infusion for a time period of between 2 and 48 hours, more preferably for a period of between 6 and 24 hours. During this period, the plasma exposure level (steady state concentration) of the active ingredient is preferably between 100 nanomolar and 5 micromolar, more preferably between 300 nanomolar to 3 micromolar. This period of intravenous treatment may then be followed by a period of peroral dosing, for example the patient may receive a dose once daily to three times daily, with daily dosing totalling between 100 mg and 1500 mg (for example between 200 mg and 1200 mg, such as from about 250 mg to about 1000 mg, e.g. from about 300 mg to about 800 mg), so as to achieve an average plasma exposure level (concentration average) between 100 nanomolar and 5 micromolar, more preferably between 300 nanomolar to 3 micromolar. PO treatment may continue for up to 30 days (for example from 1 to 30 days, such as from 2 to 30 days, e.g. from 2 to 20 days or 2 to 10 days (in particular, the peroral dosing period may be 3 days).

In particular embodiments of the uses and methods described herein (in particular, those in which the patient has and/or is being treated for a myocardial infarction (including, particularly, ST-elevation myocardial infarction), the treatment comprises administration of the compound of formula I, or pharmaceutically acceptable salt thereof, before reperfusion of the occluded vessel (wherein reperfusion is preferably achieved by treatment with percutaneous coronary intervention), and, optionally (preferably), continuing to administer the compound of formula I, or pharmaceutically acceptable salt thereof, during and/or (e.g. and) after reperfusion of the occluded vessel. In such embodiments, it may be understood that treatment with the compound of formula I, or pharmaceutically acceptable salt thereof, begins before reperfusion of the occluded vessel (e.g. with percutaneous coronary intervention) then continues during and after percutaneous coronary intervention.

Administration of a compound of formula I, or a pharmaceutically acceptable salt thereof, in a patient with and/or being treated for a myocardial infarction (including, particularly, an ST-elevation myocardial infarction) should preferably be commenced shortly before or shortly after reperfusion of the occluded vessel (e.g. by PCI). For example, administration may be commenced in the time period of from 6 hours before to 24 hours after reperfusion of the occluded vessel. Without wishing to be bound by theory, treatment within this window is believed to prevent thrombus formation and/or thrombus growth in the heart (including, particularly, left ventricular thrombus formation/growth), intramyocardial haemorrhage and/or expansion of the infarct size, as treatment with the compounds of the invention is believed to reduce the risk of reperfusion injury occurring and/or to minimise the severity of any such injury.

More preferably, treatment with a compound of formula I, or a pharmaceutically acceptable salt thereof, should be commenced before reperfusion of the occluded vessel, such as about 6 hours or less before reperfusion, more particularly, about 4 hours or less before reperfusion, such as about 2 hours or less before reperfusion, e.g. about 1 hour or less before reperfusion (or about 10 minutes or less before reperfusion (e.g. about 5 minutes or less before reperfusion). Administration within this window should ensure delivery of the active compound to the heart tissue before reperfusion of the vessel, which is further believed to reduce the risk of reperfusion injury occurring and/or to minimise the severity of any such injury.

In particular embodiments that may be mentioned, treatment with a compound of formula I, or a pharmaceutically acceptable salt thereof, is commenced from about 1 hour before to about 2 minutes before reperfusion of the occluded vessel (e.g. from about 30 minutes before to about 2 minutes before reperfusion of the occluded vessel). Initial administration of a compound of formula I, or pharmaceutically acceptable salt thereof, may particularly be peroral or intravenous. More particularly, administration shortly before and/or shortly after reperfusion of the occluded vessel may be intravenous to ensure rapid delivery of the active compound to the heart tissue.

Administration of the compound of formula I, or pharmaceutically acceptable salt thereof, then preferably continues for a period of at least 24 hours after reperfusion of the occluded vessel, such as for a period of at least 48 hours after reperfusion of the occluded vessel (e.g. at least 72 hours after reperfusion of the occluded vessel). More particularly, administration of the compound of formula I, or pharmaceutically acceptable salt thereof, continues for a period of at least 72 hours following reperfusion of the occluded vessel (e.g. by PCI).

Provided that the compound of formula I, or pharmaceutically acceptable salt thereof, is administered for at least the relevant minimum period after reperfusion of the occluded vessel (such as for at least 72 hours after reperfusion), the total duration of the treatment with the compound of formula I, or pharmaceutically acceptable salt thereof, may be determined by the skilled person as appropriate. For example, administration of the compound of formula I, or pharmaceutically acceptable salt thereof, may continue after reperfusion of the occluded vessel for a period of up to 1 month, 2 weeks or 1 week (168 hours) (for example, the period of administration after reperfusion may be for a period of from about 24 hours to about 168 hours, such as from about 48 hours to about 168 hours, e.g. from about 72 hours to about 168 hours). Such administration may be via any route, and particularly includes intravenous and/or peroral administration. More particularly, the compound of formula I, or pharmaceutically acceptable salt thereof, may be administered intravenously during and immediately following reperfusion (e.g. for a period of up to 10 hours following reperfusion (such as up to 6 hours)) then perorally for the remainder of the treatment period.

Thus, the methods and uses described herein in which the patient has and/or is being treated for a myocardial infarction (including, particularly, ST-elevation myocardial infarction), may preferably comprise administering the compound of formula I, or pharmaceutically acceptable salt thereof, before and after treatment with percutaneous coronary intervention. For the avoidance of doubt, such embodiments may also include administration of the compound of formula I, or pharmaceutically acceptable salt thereof, during treatment with percutaneous coronary intervention.

In further particular embodiments in which the patient being treated has and/or is being treated for a myocardial infarction (including an ST-elevation myocardial infarction), administration, of the compound of formula I, or pharmaceutically acceptable salt thereof, to the patient in need thereof is commenced at a timepoint within about 4 hours before reperfusion of the occluded vessel (wherein reperfusion is preferably achieved by treatment with percutaneous coronary invention) to about 24 hours after reperfusion of the occluded vessel. More particularly, administration (including, particularly, intravenous administration) of the compound of formula I, or pharmaceutically acceptable salt thereof, is commenced at a time point within from about 2 hours before reperfusion of the occluded vessel (e.g. by PCI) to about 6 hours after reperfusion (e.g. by PCI). Most particularly, administration of the compound of formula I, or pharmaceutically acceptable salt thereof, (including, particularly, intravenous administration) is commenced at a time point within about 2 hours, or preferably 1 hour, (such as 2 minutes before) before reperfusion of the occluded vessel (e.g. by PCI).

In further particular embodiments, administration of the compound of formula I, or pharmaceutically acceptable salt thereof, is commenced from about 4 hours to about 2 minutes (such as from about 2 hours to about 2 minutes, for example from about 1 hour to about 2 minutes) before reperfusion of the occluded vessel (e.g. by PCI) and continues for a period of at least about 24 hours (such as from about 24 hours to about 168 hours), particularly at least about 48 hours (such as from about 48 hours to about 168 hours), more particularly for at least about 72 hours (such as from about 72 hours to about 168 hours) after reperfusion of the occluded vessel (e.g. by PCI). In a particular embodiment that may be mentioned, administration of the compound of formula I, or pharmaceutically acceptable salt thereof, begins (preferably intravenously), from about 1 hour to about 2 minutes before reperfusion of the occluded vessel (e.g. by PCI), and continues for a period of at least 72 hours after reperfusion. More particularly, the compound of formula I, or pharmaceutically acceptable salt thereof, is administered intravenously during reperfusion and up to 10 hours (e.g. hours) following reperfusion and then orally for the remainder of the treatment period.

In more particular embodiments, the methods described herein, which the patient has and/or is being treated for a myocardial infarction (including, particularly, ST-elevation myocardial infarction comprise administration of an intravenous bolus dose of the compound of formula I, or pharmaceutically acceptable salt thereof, prior to reperfusion of the occluded vessel (e.g. by percutaneous coronary intervention) (which may be administered in the time period beginning 1 hour to 2 minutes before reperfusion), followed by intravenous infusion of the compound of formula I, or pharmaceutically acceptable salt thereof, optionally for a period of at least about 1 hour, such as about 1 hour to about 12 hours, for example about 3 hours to about 9 hours (e.g. about 6 hours). In further particular embodiments, the intravenous infusion of the compound of formula I, or pharmaceutically acceptable salt thereof, is followed by oral administration of the compound of formula I, or pharmaceutically acceptable salt thereof, for a period of at least about 24 hours, such as from about 24 hours to about 168 hours. In particular, for a period of about 72 hours.

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “+10%”, or the like (or by indicating a variance of a specific amount calculated based on the relevant value). It is also contemplated that, at each instance, such terms may be deleted.

For the avoidance of doubt, the skilled person (e.g. the physician) will be able to determine the actual dosage which will be most suitable for an individual patient, which is likely to vary with the route of administration, the type and severity of the condition that is to be treated, as well as the species, age, weight, sex, renal function, hepatic function and response of the particular patient to be treated. Although the above-mentioned dosages are exemplary of the average case, there can, of course, be individual instances where higher or lower dosage ranges are merited, and such doses are within the scope of the invention.

### Combinations and Kits-of-Parts

As described herein, compounds of the invention may also be combined with one or more other (i.e. different) therapeutic agents (i.e. agents that are not compounds of the invention) that are useful in the prevention of thrombus formation (including all aspects thereof, and the related indications described herein). Such combination products that provide for the administration of a compound of the invention in conjunction with one or more other therapeutic agent may be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the one or more other therapeutic agent).

Thus, according to a further aspect of the invention, there is provided a combination product comprising:
(I) a compound of formula I (as defined herein), or a pharmaceutically acceptable salt thereof;
(II) at least one antiplatelet medicament (e.g. as defined herein); and, optionally,
(III) at least one anticoagulant (e.g. as defined herein) wherein each of components (I) and (II) (and if present (III)) is formulated in admixture, optionally with one or more a pharmaceutically-acceptable excipient.

Thus, according to a further aspect of the invention, there is provided a combination product comprising:
(I) a compound of formula I (as defined herein), or a pharmaceutically acceptable salt thereof;
(II) at least one anticoagulant (e.g. as defined herein); and, optionally,
(III) at least one antiplatelet medication (e.g. as defined herein) wherein each of components (I) and (II) (and if present (III)) is formulated in admixture, optionally with one or more a pharmaceutically-acceptable excipient.

In a further aspect of the invention, there is provided a kit-of-parts comprising:
(a) a compound of formula I (as defined herein), or a pharmaceutically acceptable salt thereof; and
(b) at least one antiplatelet medication (e.g. as defined herein); and, optionally
(c) at least one anticoagulant (e.g. as defined herein)
which components (a) and (b) (and, if present (c)) are each provided in a form that is suitable for administration in conjunction with the other.

In a further aspect of the invention, there is provided a kit-of-parts comprising:
(a) a compound of formula I (as defined herein), or a pharmaceutically acceptable salt thereof; and
(b) at least one anticoagulant (e.g. as defined herein); and, optionally
(c) at least one antiplatelet medication (e.g. as defined herein)
which components (a) and (b) (and, if present (c)) are each provided in a form that is suitable for administration in conjunction with the other.

Such combination products and kits of parts may be used in the prevention of thrombus formation, and other related indications, described herein.

### Preparation of Compounds/Compositions

Pharmaceutical compositions/formulations, combination products and kits as described herein may be prepared in accordance with standard and/or accepted pharmaceutical practice.

Thus, in a further aspect of the invention there is provided a process for the preparation of a pharmaceutical composition/formulation, as hereinbefore defined, which process comprises bringing into association a compound of the invention, as hereinbefore defined, with one or more pharmaceutically-acceptable excipient.

In further aspects of the invention, there is provided a process for the preparation of a combination product or kit-of-parts as hereinbefore defined, which process comprises bringing into association a compound of the invention, as hereinbefore defined, with the other therapeutic agent that is useful in the treatment of the relevant disease or disorder, and at least one pharmaceutically-acceptable excipient.

As used herein, references to bringing into association will mean that the two components are rendered suitable for administration in conjunction with each other.

Thus, in relation to the process for the preparation of a kit-of-parts as hereinbefore defined, by bringing the two components “into association with” each other, we include that the two components of the kit-of-parts may be:
(i) provided as separate formulations (i.e. independently of one another), which are subsequently brought together for use in conjunction with each other in combination therapy; or
(ii) packaged and presented together as separate components of a “combination pack” for use in conjunction with each other in combination therapy.

Compounds of the invention as described herein may be prepared in accordance with techniques that are well known to those skilled in the art. In particular, compounds of formula I may be prepared in accordance with the procedures described in Karlström et al. J. Med. Chem., 2013, 56, 3177-3190; WO 2019/219771 and WO 2020/008064 and/or by conventional synthetic procedures, in accordance with standard techniques, from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3rd edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

As in all conventional organic synthesis, the functional groups of intermediate compounds may need to be protected by protecting groups in the course of the synthetic route. The protection and deprotection of functional groups may take place before or after a reaction in the above-mentioned schemes.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3^{rd} edition, T. W. Greene & P. G. M. Wutz, Wiley-Interscience (1999), the contents of which are incorporated herein by reference.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

### Further Embodiments of the Invention

The invention will be further defined in the following numbered paragraphs.

Paragraph 1. A compound of formula I,

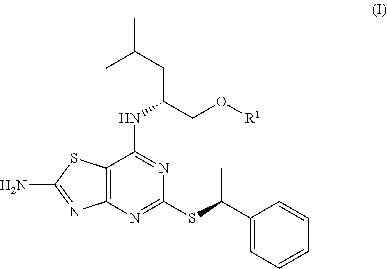

wherein R¹ is selected from hydrogen and —PO(OR²)(OR³),
wherein R² and R³ are each independently selected from the group consisting of hydrogen, C₁₋₄ alkyl and C₂₋₄ alkenyl, or a pharmaceutically acceptable salt thereof,
for use in the prevention of thrombus formation and/or thrombus growth.

Paragraph 2. A method of preventing thrombus formation and/or thrombus growth comprising administering a therapeutically effective amount of a compound of formula I, or a pharmaceutically acceptable salt thereof, as defined in Paragraph 1, to a patient in need thereof.

Paragraph 3. The use of a compound of formula I, or a pharmaceutically acceptable salt thereof, as defined in Paragraph 1, in the manufacture of a medicament for the prevention of thrombus formation and/or thrombus growth.

Paragraph 4. The compound for use, method or use as defined in any one of Paragraphs 1 to 3, wherein the compound of formula I is selected from the group consisting of

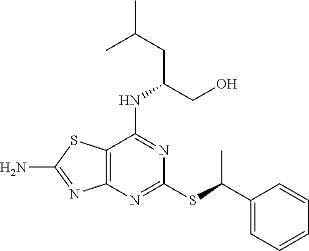 [SMILES: CC(C)C[C@H](CO)Nc1nc(S[C@@H](C)c2ccccc2)nc2nc(N)sc12]

(2R)-2-[(2-amino-5-{[(1S)-1-phenylethyl]thio}[1,3]thiazolo[4,5-d]pyrimidin-7-yl)amino]-4-methylpentan-1-ol; and

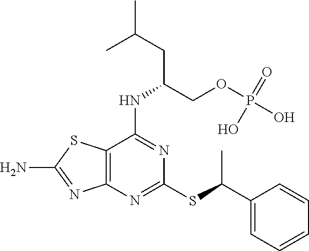 [SMILES: CC(C)C[C@H](COP(=O)(O)O)Nc1nc(S[C@@H](C)c2ccccc2)nc2nc(N)sc12]

(2R)-2-[(2-Amino-5-{[(1S)-1-phenylethyl]sulfanyl}[1,3]thiazolo[4,5-d]pyrimidin-7-yl)amino]-4-methylpentyl dihydrogen phosphate;
   or a pharmaceutically acceptable salt thereof.

Paragraph 5. The compound for use, method or use as defined in any one of Paragraphs 1 to 4, wherein the thrombus is in the heart.

Paragraph 6. The compound for use, method or use as defined in any one of Paragraphs 1 to 5, wherein the thrombus is a left ventricular thrombus.

Paragraph 7. The compound for use, method or use as defined in any one of Paragraphs 1 to 6, wherein the prevention of thrombus formation and/or thrombus growth is in a patient with and/or being treated for a myocardial infarction, particularly an acute myocardial infarction.

Paragraph 8. The compound for use method or use as defined in Paragraph 7, wherein the myocardial infarction is an ST-elevation myocardial infarction.

Paragraph 9. The compound for use, method or use as defined in any one of Paragraphs 1 to 8, wherein the myocardial infarction is an anterior myocardial infarction, optionally wherein the patient has a left ventricular ejection fraction of less than 50%.

Paragraph 10. The compound for use, method or use as defined in any one of Paragraphs 7 to 9, wherein the use or method further comprises preventing or suppressing expansion of myocardial infarct size.

Paragraph 11. The compound for use, method or use as defined in any one of Paragrpahs 1 to 10, wherein the prevention of thrombus formation and/or thrombus growth is in a patient with dilated cardiomyopathy and/or heart failure.

Paragraph 12. The compound for use, method or use as defined in any one of Paragraphs 1 to 11, wherein the compound of formula I, or pharmaceutically acceptable salt thereof, also reduces the risk of haemorrhage.

Paragraph 13. The compound for use, method or use as defined in Paragraph 12, wherein the haemorrhage is in the CNS or in a vital organ.

Paragraph 14. The compound for use, method or use as defined in Paragraph 12 or Paragraph 13, wherein the haemorrhage is intramyocardial haemorrhage.

Paragraph 15. The compound for use, method or use as defined in Paragraph 14, wherein the patient is a patient with and/or being treated for myocardial infarction, and wherein the use or method further comprises preventing or suppressing expansion of myocardial infarct size.

Paragraph 16. The compound for use, method or use as defined in Paragraph 12 or Paragraph 13, wherein the haemorrhage is a haemorrhagic stroke or a haemorrhagic transformation of an ischemic stroke; optionally wherein the haemorrhage is a haemorrhagic stroke

Paragraph 17. The compound for use, method or use, as defined in any one of Paragraphs 1 to 16, wherein the prevention of thrombus formation and/or thrombus growth is in a patient undergoing reperfusion therapy, optionally wherein the patient is undergoing reperfusion therapy for ST-elevation myocardial infarction or large vessel occlusion stroke.

Paragraph 18. The compound for use, method or use, as defined in any one of Paragraphs 1 to 17, wherein the prevention of thrombus formation and/or thrombus growth further comprises prevention of reperfusion injury.

Paragraph 19. The compound for use, method or use, as defined in any one of Paragraphs 1 to 18, wherein the patient has been treated with or is being treated with percutaneous coronary intervention.

Paragraph 20 The compound for use, method or use as defined in any one of Paragraphs 1 to 19, wherein the prevention of thrombus formation and/or thrombus growth further comprises the prevention of arterial thrombosis.

Paragraph 21. The compound for use, method or use as defined in any one of Paragraphs 1 to 19, wherein the prevention of thrombus formation and/or thrombus growth further comprises the prevention of venous thrombosis.

Paragraph 22. The compound for use, method or use as defined in any one of Paragraphs 1 to 19, wherein the prevention of thrombus formation and/or thrombus growth further comprises the prevention of microvascular thrombosis.

Paragraph 23. The compound for use, method or use as defined in any one of Paragraphs 1 to 22, wherein the prevention of thrombus formation and/or thrombus growth further comprises the prevention of systemic embolism.

Paragraph 24. The compound for use, method or use as defined in any one of Paragraphs 1 to 23, wherein the prevention of thrombus formation and/or thrombus growth further comprises the prevention of cardioembolic stroke.

Paragraph 25. A compound of formula I, or a pharmaceutically acceptable salt thereof, as defined in Paragraph 1 or Paragraph 4, for use in the prevention of haemorrhage.

Paragraph 26. The compound for use as defined in Paragraph 25, wherein the haemorrhage is as defined in any one of Paragraphs 13 to 16.

Paragraph 27. The compound for use as defined in Paragraphs 25 or 26, wherein the haemorrhage is intramyocardial haemorrhage.

Paragraph 28. The compound for use as defined in any one of Paragraphs 25 to 27, wherein the haemorrhage is in patient with and/or being treated for myocardial infarction, optionally wherein the myocardial infarction is an ST-elevation myocardial infarction.

Paragraph 29. The compound for use as defined in Paragraph 28, wherein the use further comprises the prevention or suppression of expansion in myocardial infarct size.

Paragraph 30. The compound for use as defined in any one of Paragraphs 25 to 29, wherein the compound is also for use in preventing thrombus formation and/or thrombus growth, as defined in any one of Paragraphs 1 to 10 or 17 to 24.

Paragraph 31. A compound of formula I, or a pharmaceutically acceptable salt thereof, as defined in Paragraph 1 or Paragraph 4, for use in preventing or suppressing expansion of myocardial infarct size in a patient with and/or being treated for myocardial infarction.

Paragraph 32. The compound for use as defined in Paragraph 31, wherein the use is in a patient without intramyocardial haemorrhage.

Paragraph 33. The compound for use as defined in Paragraph 31, wherein the use further comprises preventing intramyocardial haemorrhage.

Paragraph 34. The compound for use as defined in any one of Paragraphs 31 to 33, wherein the myocardial infarction is an ST-elevation myocardial infarction.

Paragraph 35. A compound of formula I, or a pharmaceutically acceptable salt thereof, as defined in Paragraph 1 or Paragraph 4 for use in the treatment of a cardiovascular disease selected from the group consisting of ST-elevation myocardial infarction, heart failure, and dilated cardiomyopathy, wherein the treatment further comprises prevention of thrombus formation and/or thrombus growth.

Paragraph 36. A method of treating a cardiovascular disease selected from the group consisting of ST-elevation myocardial infarction, heart failure, and dilated cardiomyopathy, comprising administering a therapeutically effective amount of a compound of formula I, or a pharmaceutically acceptable salt thereof, as defined in Paragraph 1 or Paragraph 4, to a patient need thereof, wherein the treatment further comprises prevention of thrombus formation and/or thrombus growth.

Paragraph 37. The use of a compound of formula I, or a pharmaceutically acceptable salt thereof, as defined in Paragraph 1 or Paragraph 4, for the manufacture of a medicament for the treatment of a cardiovascular disease selected from the group consisting of ST-elevation myocardial infarction, heart failure, and dilated cardiomyopathy, wherein the treatment further comprises prevention of thrombus formation and/or thrombus growth.

Paragraph 38. The compound for use, method or use as defined in Paragraphs 35 to 37, wherein the treatment further comprises prevention of haemorrhage, optionally wherein the haemorrhage is as defined in any one of Paragraphs 12 to 16, such as intramyocardial haemorrhage.

Paragraph 39. The compound for use, method or use as defined in any one of Paragraphs 1 to 38, wherein the compound of formula I, or pharmaceutically acceptable salt thereof is administered in conjunction with at least one antiplatelet medication.

Paragraph 40. The compound for use, method or use as defined in any Paragraph 39, wherein the at least one antiplatelet medication is selected from the group consisting of a P2Y₁₂ receptor inhibitor an adenosine reuptake inhibitor, a glycoprotein IIb/IIIa inhibitor, an (irreversible) cyclooxygenase inhibitor, a phosphodiesterase inhibitor, a protease-activated receptor-1 antagonist, a thromboxane inhibitor, a thromboxane receptor antagonist and a thromboxane synthase inhibitor, and combinations thereof.

Paragraph 41. The compound for use, method or use as defined in Paragraph 40, wherein the at least one antiplatelet medication is selected from the group consisting of a P2Y₁₂ receptor inhibitor, an (irreversible) cyclooxygenase inhibitor, a glycoprotein IIb/IIIa inhibitor, and combinations thereof.

Paragraph 42. The compound for use, method or use as defined in any one of Paragraphs 39 to 41, wherein the at least one antiplatelet medication comprises an (irreversible) cyclooxygenase inhibitor and/or a P2Y₁₂ receptor inhibitor, and, optionally, a glycoprotein IIb/IIIa inhibitor.

Paragraph 43. The compound for use, method or use as defined in Paragraph 42, wherein the (irreversible) cyclooxygenase inhibitor is aspirin, the P2Y₁₂ receptor inhibitor is prasugrel and/or the glycoprotein IIb/IIIa inhibitor is tirofiban.

Paragraph 44. The compound for use, method or use as defined in any one of Paragraphs 1 to 43, wherein the compound of formula (I), or pharmaceutically acceptable salt thereof, is administered in conjunction with at least one anticoagulant.

Paragraph 45. The compound for use, method or use as defined in Paragraph 44, wherein the at least one anticoagulant is selected from the group consisting of a vitamin K antagonist, a heparin, a synthetic pentasaccharide inhibitor of factor Xa, a direct factor Xa inhibitor, a direct thrombin inhibitor, and combinations thereof.

Paragraph 46. The compound for use, method or use as defined in Paragraph 45, wherein the at least one anticoagulant is unfractionated heparin or a low molecular weight heparin.

Paragraph 47. The compound for use, method or use as defined in any one of Paragraphs 1 to 46, wherein the compound of formula I, or pharmaceutically acceptable salt thereof, is administered in conjunction with at least one antiplatelet medication and at least one anticoagulant.

Paragraph 48. The compound for use, method or use as defined in any one of Paragraphs 1 to 47, wherein the at least one antiplatelet medication is as defined in any one of Paragraphs 40 to 43 and/or the at least one anticoagulant is as defined in Paragraph 45 or 46.

Paragraph 49. A combination product comprising:
a. a compound of formula I, or a pharmaceutically acceptable salt thereof, as defined in Paragraph 1 or Paragraph 4;
b. at least one antiplatelet medication, optionally as defined in any one of Paragraphs 40 to 43; and, optionally,
c. at least one anticoagulant, optionally as defined in any one of Paragraphs or 46.

Paragraph 50. A combination product comprising:
a. a compound of formula I, or a pharmaceutically acceptable salt thereof, as defined in Paragraph 1 or Paragraph 4;
b. at least one anticoagulant, optionally as defined in any one of Paragraphs or 46; and, optionally,
c. at least one antiplatelet medication, optionally as defined in any one of Paragraphs 40 to 43.

Paragraph 51. The combination product as defined in Paragraph 49 or Paragraph 50, for use in preventing thrombus formation and/or thrombus growth, preventing intramyocardial haemorrhage or treating a cardiovascular disease, as defined in any one of Paragraphs 1 to 38.

Without wishing to be bound by theory, it is believed that the compounds for use, methods, uses and combination therapies described herein, have the advantage of significantly reducing the risk of thrombus formation, particularly in myocardial infarction patients, and other patients at high risk of developing left ventricular thrombus, and also that the patients can prevent haemorrhage in the same patient groups. More particularly, the compounds of formula I, as described herein, (including pharmaceutically acceptable salts thereof), can cause a reduction in both left ventricular thrombus formation and intramyocardial haemorrhage in myocardial infarction, leading to a significant protective effect on the myocardium, potentially to include reducing the risk of adverse cardiovascular remodelling, ventricular arrhythmias, systemic embolism and heart failure (acute (including cardiogenic shock and chronic heart failure). Further, the compounds of the invention have been found to produce a significant reduction in left ventricular thrombus formation and intramyocardial haemorrhage when used in conjunction with established antiplatelet and anticoagulant therapies, in comparison with the use of the established therapies in the absence of the compounds of the invention.

Further, it is believed that these combined properties of the compounds of the invention described herein make them useful in treating patients undergoing reperfusion treatment and should have the effect of providing improved protection of vital organ function.

### BRIEF DESCRIPTION OF THE FIGURES

FIG. 1 shows an overview of the clinical study described in the Examples.

### EXAMPLES

The present invention will be further described by reference to the following example, which is not intended to limit the scope of the invention.

### Clinical Study

The FRACTAL (FRACTalkine inhibition in Acute myocardial infarction) study was designed as a phase Iia, randomised, two-arm parallel-group, placebo-controlled, double-blind, multi-centre trial. The trial was primarily intended to assess the safety tolerability of intravenous and oral administration of (2R)-2-[(2-amino-5-{[(1S)-1-phenylethyl]thio}[1,3]thiazolo[4,5-d]pyrimidin-7-yl)amino]-4-methylpentan-1-ol (KAND567) (hydrochloride salt) (′investigational medicinal product (IMP)′; structure below)

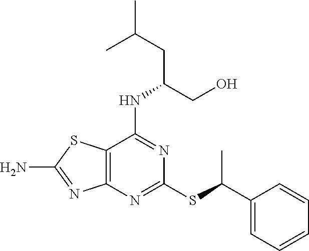 [SMILES: CC(C)C[C@H](CO)Nc1nc(S[C@@H](C)c2ccccc2)nc2nc(N)sc12]

in ST-elevation myocardial infarction (STEMI) patients undergoing percutaneous coronary intervention. For this, a cohort of 71 STEMI patients were admitted into two large tertiary centres in the United Kingdom. Secondary endpoints included investigating the anti-inflammatory and cardioprotective effects of the treatment.

Included were patients within 5 h of chest pain onset with new anterior ST segment elevation, and an angiographic image confirming an occlusion in the proximal or mid-left anterior descending coronary artery (LAD) with Thrombolysis in Myocardial Infarction (TIMI) flow of 0-2. Only patients between the ages of 18 to 75 years old were accepted into the study. Exclusion criteria included cardiogenic shock, previous MI in the LAD territory, documented previous left ventricular systolic dysfunction (ejection fraction <40%), previous coronary artery bypass graft (CABG), current use of steroids, immunosuppression, or benzodiazepines, active malignancy, renal failure, hepatic failure, and patients with any contraindication to cardiac MRI (CMR) scanning. Arterial blood sampling was obtained at the start of primary percutaneous coronary intervention (PPCI).

Full details of inclusion or exclusion criteria are provided below.

### Inclusion Criteria

1. Reperfusion of anterior MI expected within 5 hours of onset of acute chest pain.
2. Diagnosis of acute myocardial infarction with ST elevation at the J-point in two contiguous leads by ECG (V1-V4 at least two contiguous leads STE 2.0 mm in men and 1.5 mm in women).
3. Agreement to undergo routine procedure of Primary Percutaneous Coronary Intervention (PPCI).
4. Aged ≥18-75 years
5. Willingness and ability to comply with trial procedures, visit schedules, trial restrictions and requirements.
6. Confirmation of anterior STEMI (TIMI 0-2 flow) by angiography and occlusion of proximal or mid LAD (segments 5 or 6).
7. Verbal consent to take part in the trial is given.
   Exclusion Criteria 1. Anyone with known cognitive impairment or unable to provide verbal consent.
2. Serious co-existing medical condition, including known hepatic failure and known renal failure with known eGFR <30 mL/min/m2 or severe mental disorder, known at the time of randomisation.
3. Cardiogenic shock, non-compensated acute heart failure and/or pulmonary edema.
4. Previous known major vascular intervention within the last 4 weeks.
5. History of previous myocardial infarction (MI) in the Left Anterior Descending Artery (LAD).
6. Documented left ventricular systolic dysfunction (EF<40%).
7. Previous coronary artery bypass grafting (CABG)
8. Patients unable to tolerate or undergo MRI scanning including patients with claustrophobia, cardiac pacemaker/defibrillator, ferromagnetic metal implants unless approved for use in MRI scanners or excessive body weight.
9. Known planned hospitalisations (e.g. elective surgery), or other scheduled treatment for pre-existing conditions during the course of the trial that could interfere with clinical assessment.
10. Known allergy to contrast agent or other contraindications for angiography.
11. Any known previous diagnosis of invasive cancer within the last 5 years except for treated basal cell carcinoma of the skin.
12. Current use of steroids or immunosuppressants.
13. Current use of benzodiazepines.
14. Current use of ticagrelor (unable to switch to Prasugrel on trial entry).
15. Known pre-existing severe liver disease, including chronic hepatitis or alcohol-dependent liver cirrhosis
16. Other medical or social reasons for exclusion at the discretion of the investigator
17. Administration of any investigational drug within 3 months of trial medication, as far as known to patient.
18. Current use of drug sensitive to CYP3A4 inhibition which cannot be paused or switched to an alternative from the same class of medication for the period of IMP administration, including:
a) Certain P2Y₁₂ inhibitors (clopidogrel)
b) Certain statins (lovastatin and simvastatin)
19. Women of child-bearing potential who are sexually active who are not using a contraceptive method with a failure rate of <1% to prevent pregnancy prior to trial entry (copper intrauterine device, levonorgestrel-releasing intrauterine system (LNG-IUS), progesterone implant, bilateral tube occlusion or hysterectomy, vasectomised partner (>3 months earlier), sexual abstinence). Postmenopausal women must be amenorrhoeic for at least 12 months prior to randomisation to be considered of non-childbearing potential.
20. Women of child-bearing potential who are not willing to use contraceptive method with a failure rate of <1% to prevent pregnancy throughout the trial (as 19.)
21. Male patients with female partners of childbearing potential not willing to use effective contraception as detailed in section (Male participants: condom, sexual abstinence, vasectomy; female partners: combined (estrogen and progesterone-containing) hormonal contraception associated with inhibition of ovulation, progesterone-only hormonal contraceptive associated with inhibition of ovulation, IUD, intrauterine hormone-releasing system, bilateral tube occlusion or hysterectomy). 22. Male patients must agree to refrain from donating sperm whilst participating in the trial.

### Procedure

Following angiography and final confirmation of eligibility, patients were randomised to receive the intravenous (IV) bolus of either investigational medicinal product (IMP) or placebo. To avoid delivery of the IMP after reperfusion of the occluded vessel, percutaneous coronary intervention (PCI) was only commenced following the intravenous bolus of IMP/placebo. Patients then completed the remainder of their IMP infusion over 6 h. Following the completion of their intravenous infusion, patients in both arms continued an oral IMP/placebo regimen over 72 h.

### Assessment of End Points

The primary outcome of the study was safety, but a number of key secondary outcomes being assessed in parallel. Blood samples at nine different time points were used to assess any changes in the immune compartment as well as in inflammatory markers, and the cardioprotective effects of KAND567, as measured by gadolinium-contrast Cardiac Magnetic Resonance (CMR) (Cardiac MRI), were assessed as an additional secondary end point.

Every patient received a baseline CMR scan on day 3 post myocardial infarction, followed by a subsequent scan after 3 months. Image analysis was performed by a Core lab which was blinded to the treatment assignment.

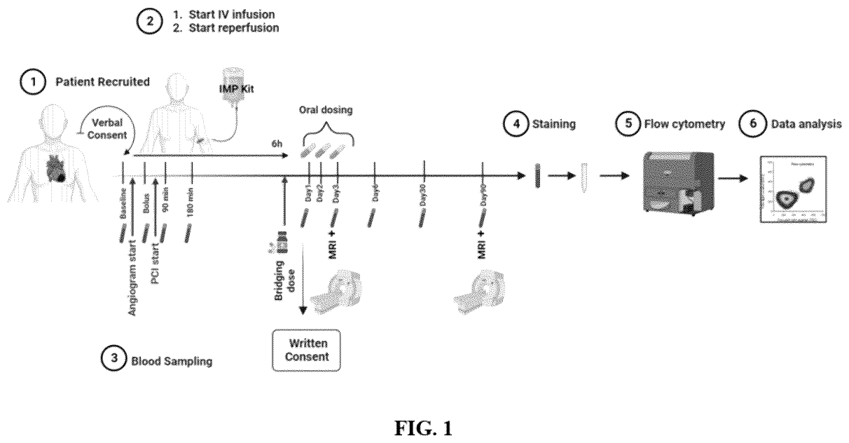

An overview of the treatment and analysis is shown in FIG. 1.

### Concomitant Medications

The clinical trial protocol allowed for the use of the following medications before, during and after percutaneous coronary intervention, as shown in the Table below.

| Before PCI | During PCI | After PCI |
|---|---|---|
| Aspirin (lifelong) | Heparin (Fragmin ®) | Beta blocker (e.g. |
| Prasugrel (continued | Tirobifan (optional) | metoprolol, bisoprolol) |
| for 1 year) | Nitroglycerin and | (lifelong) |
| Analgesia (morphine | adenosine | Statins (e.g. |
| or fentanyl) | Optionally) | atorvastatin) (lifelong) |
| | metoclopramide | Prasugrel (continued |
| | (antiemetic) or | for 1 year) |
| | ordanestron | Eplerenone (if EF <40% |
| | | and clinical left |
| | | ventricular failure |
| | | (LVF) |
| | | ACE inhibitor (e.g. |
| | | ramipril) (lifelong) |
| | | Diuretics (furosemide) |
| | | (if heart failure) |

### Antiplatelet and Anticoagulant Medications

### Antiplatelet

all patients received aspirin and prasugrel (P2Y₁₂ inhibitor)
Approximately 50% of patients (52.9% placebo group; 52.8% IMP group) received tirofiban (glycoprotein IIb/IIIa inhibitor).

### Anticoagulant

All patients received heparin (periprocedural)

### Dosing

The IMP and placebo (IMP 7.5 mg/mL solution (comprising 7.5 mg/mL IMP, Kleptose HP, sodium chloride and disodium phosphate in water for injection) added to 250 ml sodium chloride 0.9%, placebo solution as IMP solution but without active ingredient) were administered intravenously over a 6 hour period, at the rate summarised in the table below.

Summary of intravenous administration*

| Intravenous infusions | | KAND567 | Placebo |
|---|---|---|---|
| Infusion part 1 (bolus) | Time (min) | 2.0 | 2.0 |
| 0 mins-2 mins | Rate (ml/hour) | 555 | 555 |
| | Volume (ml) | 18.5 | 18.5 |
| | Amount IMP (mg) | 10 | 0.0 |
| Infusion part 2 | Time (min) | 178 | 178 |
| (maintenance) | | | |
| 2 mins-3 hours | Rate (ml/hour) | 42.5 | 42.5 |
| | Volume (ml) | 126 | 126 |
| | Amount IMP (mg) | 68 | 0.0 |
| Infusion part 3 | Time (min) | Until infusion bag fully | |
| (maintenance) | | infused** | |
| 3 hours-6 hours | Rate (ml/hour) | 35 | 35 |
| | Volume (ml) | 104 | 104 |
| | Amount IMP (mg) | 56 | 0.0 |
| Total infusion time | | 360*** | 360*** |
| (min) | | | |
| Total volume (ml) | | 248.5 | 248.5 |
| Left in infusion | | 20.7 | 20.7 |
| bag/line (ml) | | | |
| Amount IMP | | 134 | 0.0 |
| administered (mg) | | | |

*The table shows both active and placebo values. The trial was blinded; therefore, the information is only representative.
**infusion time may be variable
***infusion bag fully infused was taken as the timepoint that the infusion pump is unable to appropriately administer any more fluid. At this point the giving set should be disconnected from the participant. The line was not flushed prior to removal.

Infusion of the initial bolus dose (approximately 10 mg of the active ingredient) was commenced at least 2 minutes before initiation of PCI.
Reperfusion of the occluded vessel was then initiated by advancing a coronary wire past the occluded or partially occluded site, followed by either balloon angioplasty, thrombectomy or stenting, according to routine clinical care.
IV infusion was then continued over 6 hours with an estimated steady state concentration of 1.5 μM (the rate of infusion was adjusted 3 hours after the start of the bolus dose, to achieve a steady concentration in the blood). The same IV solution was used throughout to give a total dose of 134 mg of the active ingredient over the 6-hour infusion time; placebo solution as IMP solution but without active ingredient).
If required, a bridging peroral dose of 0 to 4 capsules (50 mg capsules of IMP (white, size 1, hard capsules) or matching placebo) was administered depending on timing of presenting at the hospital. Bridging dose depended on time to next regular dose (administered at 8 am, 4 μm and 12 am) as follows:
   0-2 hours (no bridging dose)
   2-4 hours (50 mg or placebo)
   4-6 hours (100 mg or placebo)
   6-8 hours (200 mg or placebo)
Continued with PO administration (200 mg (4 capsules) or matching placebo) three times a day (8 am, 4 μm, 12 am) through 72 hours from PCI.

### Results

The IMP was found to be safe and well-tolerated.

Surprisingly, a significant reduction in left ventricular thrombus and intramyocardial haemorrhage was observed in the treatment group. (as measured by Cardiovascular Magnetic Resonance Imaging (CMR or CMRI) and/or echocardiogram (left ventricular thrombus only) on day 3 of the treatment. There was also no increased incidence of bleeding in the treatment group.

### Patients:

71 anterior STEMI patients recruited.

61 patients completing the trial through day 90 follow-up.

| Variable | Placebo | KAND567 |
|---|---|---|
| Number | 34 | 37 |
| Gender f/m | 2/32 | 5/32 |
| Age (average) | 58.38 | 59.86 |
| Weight (kg) (average) | 85.13 | 93.41 |
| Troponin (high sensitivity | 383 | 578 |
| troponin T (hsTnT)) at | | |
| baseline (ng/L) | | |

Rates of left ventricular thrombus and intramyocardial haemorrhage

| | | % with Intramyocardial |
|---|---|---|
| Study group | % with LV thrombus (N) | hemorrhage (N) |
| Placebo | 17.6 (34) | 55.2% (29) |
| IMP | 2.7 (37) | 35.5% (31) |

The observed reduction in left ventricular thrombus suggests that the investigational medicinal product, and other compounds of the invention, have a significant antithrombotic effect, even above that achieved by established ‘standard of care’ medications. Surprisingly, the compounds also achieve this antithrombotic effect without increasing the rate of bleeding in the patients, and in fact have been found to also reduce the rate of intramyocardial haemorrhage. This surprising combination of effects suggests, in addition to reducing the risk of blood clot formation, the compounds may have significant cardioprotective effects, particularly in myocardial infarction patients. These effects are also believed to lead to a reduced risk of systemic embolism, cardioembolic stroke and adverse cardiovascular remodelling, which can lead to heart failure. They also suggest that the compounds may have protective effects in patients undergoing reperfusion treatment.

Additionally, the size of the myocardial infarction was assessed using late gadolinium-enhanced (LGE) magnetic resonance imaging (MRI) at day 90 to compare infarct size in patients administered the placebo or the IMP, using a thresholding of 5 standard deviations above the mean signal intensity from normal myocardium.

The average infarct size for patients with and without intramyocardial haemorrhage in the placebo and treatment groups is shown in the table below.

| | Placebo (N) | KAND567 (N) | Statistical |
|---|---|---|---|
| Variable | Infarct size* | Infarct size* | T-test |
| with | 29.3 (15) | 30.8 (11) | p = 0.81 |
| intramyocardial | | | |
| hemorrhage | | | |
| without | 24.0 (11) | 17.8 (16) | p = 0.15 |
| intramyocardial | | | |
| hemorrhage | | | |

*GAD5-SD(%), late gadolinium-enhanced (LGE) magnetic resonance imaging (MRI) was used at day 90 to compare infarct size in patients administered placebo or KAND567 using a thresholding of 5 standard deviations above the mean signal intensity from normal myocardium.

As would be expected, intramyocardial haemorrhage was seen to be associated with a larger myocardial infarct size (Vyas et al., Journal for the Society for Cardiovascular Angiography & Interventions, 2022, 1 (6), 100444). However, it was also surprisingly found that treatment with the IMP, in addition to the expected benefit of avoiding intramyocardial hemorrhage as such, led to a further reduction in infarct size compared to the placebo group, which suggests that the compounds of the invention also have the effect of preventing and/or minimising the expansion of myocardial infarct size. This increased preservation of viable cardiac tissue is expected to translate into improved cardiovascular function and reduced major cardiovascular events such as reduced events of heart failure and cardiac death.

1. A method of preventing thrombus formation and/or thrombus growth in the heart of a patient with and/or being treated for an ST-elevation myocardial infarction, comprising administering a therapeutically effective amount of a compound of formula I, 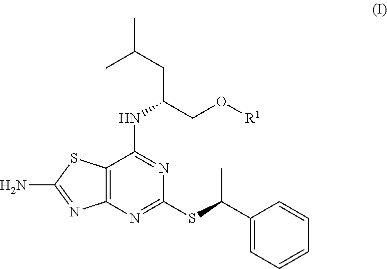
wherein R¹ is selected from hydrogen and —PO(OR²)(OR³),
wherein R² and R³ are each independently selected from the group consisting of hydrogen, C₁₋₄ alkyl and C₂₋₄ alkenyl,
or a pharmaceutically acceptable salt thereof,
to a patient in need thereof.

2. The method as claimed in claim 1, wherein the compound of formula I is selected from the group consisting of 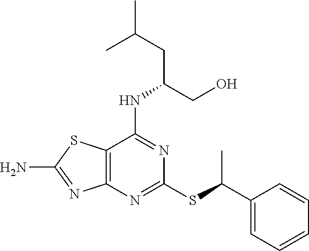 [SMILES: CC(C)C[C@H](CO)Nc1nc(S[C@@H](C)c2ccccc2)nc2nc(N)sc12]
(2R)-2-[(2-amino-5-{[(1S)-1-phenylethyl]thio}[1,3]thiazolo[4,5-d]pyrimidin-7-yl)amino]-4-methylpentan-1-ol; and 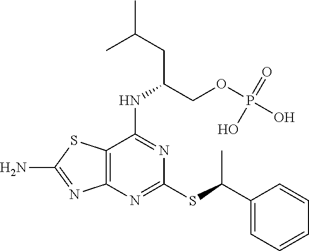 [SMILES: CC(C)C[C@H](COP(=O)(O)O)Nc1nc(S[C@@H](C)c2ccccc2)nc2nc(N)sc12]
(2R)-2-[(2-Amino-5-{[(1S)-1-phenylethyl]sulfanyl}[1,3]thiazolo[4,5-d]pyrimidin-7-yl)amino]-4-methylpentyl dihydrogen phosphate;
or a pharmaceutically acceptable salt thereof.

3. The method as claimed in claim 1, wherein the thrombus is a left ventricular thrombus.

4. The method as claimed in claim 1, wherein the myocardial infarction is an anterior ST-elevation myocardial infarction, with occlusion of the left anterior descending coronary artery.

5. The method as claimed in claim 1, wherein the patient has a left ventricular ejection fraction of less than 50%.

6. The method as claimed in claim 1, wherein the method further comprises preventing or suppressing expansion of myocardial infarct size.

7. The method as claimed in any one of claim 1, wherein the method further comprises reducing the risk of haemorrhage.

8. The method as claimed in claim 7, wherein the haemorrhage is intramyocardial haemorrhage.

9. The method as claimed in claim 8, wherein the wherein the method further comprises preventing or suppressing expansion of myocardial infarct size.

10. The method as claimed in claim 1, wherein the prevention of thrombus formation and/or thrombus growth is in a patient undergoing reperfusion therapy for ST-elevation myocardial infarction.

11. The method as claimed in claim 1, wherein the prevention of thrombus formation and/or thrombus growth further comprises prevention of reperfusion injury.

12. The method as claimed in claim 1, wherein the patient has been treated with or is being treated with percutaneous coronary intervention.

13. The method as claimed in claim 12, wherein the method comprises commencing administration the compound of formula I, or pharmaceutically acceptable salt thereof, within a time period of from about 4 hours to about 2 minutes before reperfusion of the occluded vessel by percutaneous coronary intervention, and, optionally, continuing to administer the compound of formula I, or pharmaceutically acceptable salt thereof, for a period of at least about 72 hours after percutaneous coronary intervention.

14. The method as claimed in claim 1, wherein the compound of formula I, or pharmaceutically acceptable salt thereof, is administered in conjunction with at least one antiplatelet medication.

15. The method as claimed in claim 14, wherein the antiplatelet medication is selected from the group consisting of a P2Y₁₂ receptor inhibitor an adenosine reuptake inhibitor, a glycoprotein IIb/IIIa inhibitor, an (irreversible) cyclooxygenase inhibitor, a phosphodiesterase inhibitor, a protease-activated receptor-1 antagonist, a thromboxane inhibitor, a thromboxane receptor antagonist and a thromboxane synthase inhibitor, and combinations thereof.

16. The method as claimed in claim 15, wherein the at least one antiplatelet medication comprises an (irreversible) cyclooxygenase inhibitor and/or a P2Y₁₂ receptor inhibitor, and, optionally, a glycoprotein IIb/IIIa inhibitor.

17. The method as claimed in claim 16, wherein the (irreversible) cyclooxygenase inhibitor is aspirin, the P2Y₁₂ receptor inhibitor is prasugrel and/or the glycoprotein IIb/IIIa inhibitor is tirofiban.

18. The method as claimed in claim 1, wherein the compound of formula I, or pharmaceutically acceptable salt thereof, is administered in conjunction with at least one anticoagulant.

19. The method as claimed in claim 18, wherein the at least one anticoagulant is selected from the group consisting of a vitamin K antagonist, a heparin, a synthetic pentasaccharide inhibitor of factor Xa, a direct factor Xa inhibitor, a direct thrombin inhibitor, and combinations thereof.

20. The method as claimed in claim 19, wherein the at least one anticoagulant is unfractionated heparin or a low molecular weight heparin.

21. The method as claimed in claim 1, wherein the compound of formula I is administered in conjunction with at least one antiplatelet medication and at least one anticoagulant.

22. The method as claimed in claim 21, wherein the at least one antiplatelet medication comprises an (irreversible) cyclooxygenase inhibitor and/or a P2Y₁₂ receptor inhibitor, and, optionally, a glycoprotein IIb/IIIa inhibitor and the at least one anticoagulant is selected from the group consisting of a vitamin K antagonist, a heparin, a synthetic pentasaccharide inhibitor of factor Xa, a direct factor Xa inhibitor, a direct thrombin inhibitor, and combinations thereof.

23. A method of preventing intramyocardial haemorrhage in a patient with and/or being treated for ST-elevation myocardial infarction, comprising administering a therapeutically effective amount of a compound of formula I, or a pharmaceutically acceptable salt thereof, as defined in claim 1, to a patient in need thereof.

24. The method as claimed in claim 23, wherein the method further comprises the prevention or suppression of expansion in myocardial infarct size.

25. The method as claimed in claim 23, wherein the method further comprising preventing thrombus formation and/or thrombus growth in the heart of the patient.

26. A method of preventing or suppressing expansion of myocardial infarct size in a patient with and/or being treated for ST-elevation myocardial infarction, comprising administering a pharmaceutically effective amount of a compound of formula I, or a pharmaceutically acceptable salt thereof, as defined in claim 1 to a patient in need thereof.

27. The method as claimed in claim 26, wherein the method is in a patient without intramyocardial haemorrhage.

28. The method as claimed in claim 27, wherein the method further comprises preventing intramyocardial haemorrhage.

29. A method of preventing thrombus formation and/or thrombus growth in the heart of a patient with and/or being treated for an ST-elevation myocardial infarction, comprising administering a therapeutically effective amount of a compound of formula I, or a pharmaceutically acceptable salt thereof, as defined in claim 1; wherein the treatment comprises administering the compound of formula I, or pharmaceutically acceptable salt thereof, in conjunction with at least one anti-platelet medication and at least one anti-coagulant.
